(19) United States
(12) Patent Application Publication
(10) Pub. No.: US 2023/0236192 A1
(43) Pub. Date: Jul. 27, 2023

(54) ANTI-HEPSIN ANTIBODIES AND USES THEREOF

(71) Applicant: Navaux, Inc., Little Rock, AR (US)

(72) Inventors: Blake P. Johnson, Hot Springs, AR (US); Timothy J. O'Brien, Little Rock, AR (US)

(21) Appl. No.: 17/817,539

(22) Filed: Aug. 4, 2022

Related U.S. Application Data
(63) Continuation of application No. PCT/US2021/016409, filed on Feb. 3, 2021.
(60) Provisional application No. 62/970,626, filed on Feb. 5, 2020.

(51) Int. Cl. G01N 33/573; G01N 33/574; C07K 16/40

(52) U.S. Cl. CPC G01N 33/573; G01N 33/57488; C07K 16/40; G01N 2470/04; G01N 2333/96433

(57) ABSTRACT

The present application discloses methods of making anti-hepsin antibodies, anti-hepsin antibodies, methods of screening the activity of anti-hepsin antibodies, pharmaceutical compositions of anti-hepsin antibodies, kits containing anti-hepsin antibodies, and methods of using anti-hepsin antibodies to diagnose a cancer.

### CROSS-REFERENCE

This application is a continuation of International Application No. PCT/US2021/016409, filed Feb. 3, 2021, which claims the benefit of U.S. Provisional Application No. 62/970,626 filed on Feb. 5, 2020, both of which are incorporated herein by reference in their entirety for all purposes.

### SEQUENCE LISTING

The instant application contains a Sequence Listing which has been submitted electronically in XML format and is hereby incorporated by reference in its entirety. Said XML copy, created on Jan. 25, 2023, is named 55156-701_301_SL.xml and is 58,022 bytes in size.

### BACKGROUND OF THE INVENTION

Cancer is one of the leading causes of death in the developed world and over 14 million new cases of cancer occur globally each year. Genetic and environmental factors can cause cancer and the risk of cancer increases significantly with age. Rates of cancer occurrences are increasing as people live longer and as lifestyle changes occur in the developing world. There is a need for new diagnostic methods for cancers.

### SUMMARY OF THE INVENTION

Hepsin, a type II transmembrane serine protease, is commonly overexpressed in a variety of epithelial cancers, where its overexpression and concurrent proteolytic activity correlate with tumor progression. Recent studies indicate that Hepsin undergoes activation via autocatalysis and subsequent ectodomain shedding, thus highlighting its potential as a serum-based biomarker. Using transgenic mouse models of prostate adenocarcinoma, Hepsin overexpression has previously been implicated in disease progression and, notably, metastases that are neuroendocrine in nature. Collectively, there is a need for the diagnostic utility of Hepsin expression in prostate cancer progression, inclusive of metastatic variants thereof.

Provided herein are isolated antibodies that selectively binds to circulating Hepsin or to the c-terminus of circulating hepsin. In one instance, the antibody does not selectively bind to serine proteases Matripase, KLK6, KLK7, and KLK8. In one instance, provided herein is an isolated, recombinant hepsin polynucleotide sequence that comprises SEQ ID NO: 42. In another instance, provided herein is an isolated, recombinant hepsin amino acid sequence that comprises SEQ ID NO: 43. In another instance, provided herein is an isolated, recombinant hepsin amino acid sequence that comprises SEQ ID NO: 44. In another instance, provided herein is an isolated, catalytically active, recombinant hepsin amino acid sequence that comprises SEQ ID NO: 48.

In one instance, an isolated antibody described herein may be preparable by a method of: (a) preparing hybridomas (e.g., from a rodent immunized with a recombinant Hepsin sequence that lacks a transmembrane portion (e.g., of wild-type Hepsin)); (b) screening the hybridomas of (a) against serum obtained from an individual (e.g., diagnosed with, for example, an epithelial cancer); and (c) isolating hybridomas of (b) that specifically bind to circulating (or extracellular) Hepsin.

In another instance, an isolated antibody described herein may be preparable by a method of: (a) preparing hybridomas (e.g., from a rodent immunized with a recombinant Hepsin sequence that lacks a transmembrane portion (e.g., of wild-type Hepsin)); (b) screening the hybridomas of b) against serum obtained from an individual (e.g., diagnosed with, for example, an epithelial cancer); (c) isolating hybridomas of (b) that specifically bind to extracellular Hepsin; (d) screening the hybridomas of (c) against a recombinant, biologically-active extracellular Hepsin; and (e) isolating the hybridomas of (d) that specifically bind to the extracellular, circulating c-terminal portion of Hepsin.

In some instances of the described methods, the Hepsin comprises human Hepsin. The recombinant, biologically-active extracellular Hepsin, in some instances, further comprises a thrombin cleavage site and, optionally, a spacer (linker). Circulating Hepsin may comprise an amino acid sequence of SEQ ID NO: 44. Catalytically active hepsin with a flag tag may comprise an amino acid sequence of SEQ ID NO: 48. Wild-type human Hepsin may comprise an amino acid sequence of SEQ ID NO: 41.

In one aspect, provided herein is an isolated antibody that binds to a recombinant Hepsin sequence that comprises an amino acid sequence of SEQ ID NO: 43. In some instances, binding is selective.

In one aspect, provided herein is an antibody or antigen-binding fragment thereof that comprises a variable heavy chain complementarity-determining region CDR-H1, CDR-H2 and CDR-H3, wherein CDR-H1 comprises a reconstructed polypeptide consensus sequence selected from any one of SEQ ID NOS: 16 (KYWMS), 18 (SGYSWH), and 19 (SFGMH), CDR-H2 comprises a reconstructed polypeptide consensus sequence selected from any one of SEQ ID NOS: 11 (EINPDGSTIIYTPSLKD), 13 (YIHYNGNTNYNPSLKS), and 14 (YISSGSSAIYYADTVKG), and CDR-H3 comprises a reconstructed polypeptide consensus sequence selected from any one of SEQ ID NOS: 6 (RANWYYFDY), (ADF), and 9 (SNWDYFDY).

In another aspect, provided herein is an antibody or antigen-binding fragment thereof that comprises a variable light chain complementarity-determining region CDR-L1, CDR-L2 and CDR-L3, wherein CDR-L1 comprises a reconstructed polypeptide consensus sequence selected from any one of SEQ ID NOS: 17 (TASSSVSSSNFH) and 20 (KSSQSLLNSRIRKNYLA), CDR-L2 comprises a reconstructed polypeptide consensus sequence selected from any one of SEQ ID NOS: 12 (STSNLAS) and 15 (WASTRES), and CDR-L3 comprises a reconstructed polypeptide consensus sequence selected from any one of SEQ ID NOS: 7 (HQYHRSPRT) and 10 (KQSYNLWT).

In one aspect provided herein is an antibody or antigen-binding fragment thereof that comprises: a variable heavy chain CDR-H1, CDR-H2 and CDR-H3, wherein CDR-H1 comprises a reconstructed polypeptide consensus sequence selected from any one of SEQ ID NOS: 16 (KYWMS), 18 (SGYSWH), and 19 (SFGMH), CDR-H2 comprises a reconstructed polypeptide consensus sequence selected from any one of SEQ ID NOS: 11 (EINPDGSTIIYTPSLKD), 13 (YIHYNGNTNYNPSLKS), and 14 (YISSGSSAIYYADTVKG), and CDR-H3 comprises a reconstructed polypeptide consensus sequence selected from any one of SEQ ID NOS: 6 (RANWYYFDY), (ADF), and 9 (SNWDYFDY); and wherein CDR-L1 comprises a reconstructed polypeptide consensus sequence selected from any one of SEQ ID NOS: 17 (TASSSVSSSNFH) and 20 (KSSQSLLNSRIRKNYLA), CDR-L2 comprises a reconstructed polypeptide consensus sequence selected from any one of SEQ ID NOS: 12 (STSNLAS) and 15 (WASTRES), and CDR-L3 comprises a reconstructed polypeptide consensus sequence selected from any one of SEQ ID NOS: 7 (HQYHRSPRT) and 10 (KQSYNLWT).

In one aspect, provided herein is an antibody or antigen-binding fragment thereof that comprises a variable heavy chain, wherein the variable heavy chain comprises a reconstructed polypeptide consensus sequence selected from any one of SEQ ID NOS: 1-3.

In one aspect, provided herein is an antibody or antigen-binding fragment thereof that comprises a variable light chain, wherein the variable light chain comprises a reconstructed polypeptide consensus sequence selected from any one of SEQ ID NOS: 4 and 5.

In one aspect, provided herein is an antibody or antigen-binding fragment thereof that comprises: a variable heavy chain, wherein the variable heavy chain comprises a reconstructed polypeptide consensus sequence selected from any one of SEQ ID NOS: 1-3; and a variable light chain, wherein the variable light chain comprises a reconstructed polypeptide consensus sequence selected from any one of SEQ ID NOS: 4 and 5.

In one aspect, provided herein is a hybridoma that produces the antibody or antigen-binding fragment thereof described above.

In some embodiments, the antibody comprises an IgG, IgA, or IgM antibody. In some embodiments, the IgG comprises IgG1, IgG2, IgG3, IgG4, IgGA1, or IgGA2. In some embodiments, the antibody comprises a chimeric antibody, a humanized antibody, a human antibody, a monoclonal antibody, a deimmunized antibody, a bispecific antibody, a multispecific antibody, or a combination thereof.

In some embodiments, the antibody comprises a monoclonal antibody. In some embodiments, the antibody comprises a multispecific antibody. In some embodiments, the antibody comprises a multivalent antibody. In some embodiments, the antigen-binding fragment comprises a Fab, Fab′, Fab′-SH, Fv, scFv, F(ab′)₂, a diabody, a linear antibody, a single domain antibodies (sdAb), a camelid Vi domain, or a multi-specific antibody formed from antibody fragments. In some embodiments, the antibody or antigen-binding fragment thereof is recombinant or synthetic.

In one aspect provided herein is an isolated nucleic acid that comprises a reconstructed nucleic acid consensus sequence encoding a heavy chain polypeptide of an antibody, wherein the nucleic acid consensus sequence is selected from any one of SEQ ID NOS: 21, 22, and 23.

In one aspect provided herein is an isolated nucleic acid that comprises a reconstructed nucleic acid consensus sequence encoding a light chain polypeptide of an antibody, wherein the nucleic acid consensus sequence is selected from any one of SEQ ID NOS: 24 and 25.

In one aspect provided herein is a vector that comprises the isolated nucleic acid described above. In some embodiments, isolated nucleic acid is operably linked to a regulatory control sequence.

In one aspect provided herein is a host cell that comprises the vector or the nucleic acid of any one of aspects described above.

In one aspect, provided herein is a composition comprising an antibody of any one of the preceding claims and a pharmaceutically acceptable excipient.

In one aspect, provided herein is a method of identifying the presence of circulating hepsin in a biological sample comprising: (a) contacting the biological sample (e.g., obtained from an individual, such as an individual suspected of having or at risk for cancer) with an antibody that selectively binds circulating hepsin; and (b) determining whether circulating hepsin is present in the biological sample (e.g., determining whether the amount of circulating hepsin is elevated). A biological sample may be, for example, a blood (or non-tissue) sample (e.g., whole blood, serum, urine, etc.).

In one instance, the method comprises an enzyme-linked immunosorbent assay (ELISA), enzyme-linked immune absorbent spot (ELISPOT), immunohistochemistry (IHC), antibody adaptation to microbeads for multiplex and/or microfluidic platforms, etc.

In another instance, the method further comprises identifying presence of or risk of developing cancer in an individual (e.g., the individual suspected of or at risk for cancer).

In another instance, the method further comprises identifying risk of recurrence of cancer in an individual (e.g., an individual suspected of being at risk for recurrence).

In another instance, the method further comprises identifying risk of metastasis of cancer in an individual (e.g., an individual suspected of being at risk for recurrence).

A cancer may be, for example, an epithelial cancer such as, for example, an ovarian cancer, a prostate cancer, a carcinoma, or a combination thereof. In one instance, the cancer is an ovarian cancer. In another instance, the cancer is a prostate cancer. In another instance, the cancer is a carcinoma. Non-limiting examples of carcinomas include, but are not limited to, a renal cell carcinoma (RCC) or a metastatic renal cell carcinoma.

In one aspect, provided herein is a method of treating a disorder associated with elevated levels of circulating hepsin in a subject in need thereof, comprising administering to the subject an antibody that selectively binds to circulating hepsin.

In one aspect, provided herein is a method of treating hyperhepsinemia in a subject in need thereof, comprising administering to the subject an antibody that selectively binds to circulating hepsin.

In one aspect, provided herein is a method of producing an antibody that selectively binds to circulating Hepsin, the method comprising screening hybridomas generated by immunizing an animal to hepsin against an isolated c-terminal portion of hepsin.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

The sequences described throughout the application are herein incorporated by reference.

### BRIEF DISCLOSURE OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A illustrates an exemplary assay scheme for the diagnostic methods described herein. In Step 1, sample antigen is added to a well that is coated with affinity-purified antigen specific antibodies and then incubated in Step 2 to allow for binding of antigen to the antibodies. The plate is washed, streptavidin-horseradish peroxidase (HRP) conjugate is added to the plate (Step 3), and incubated. 3,3′,5,5′-tetramethylbenzidine (TMB) substrate is added, and a stop solution is finally added following incubation. Color is detected at 45 nM.

FIG. 1B illustrates an exemplary standard curve of the results of FIG. 1A.

### DETAILED DESCRIPTION OF THE INVENTION

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Cabs, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); and The Antibodies (M. Zanetti and J. D. Capra, eds., Harwood Academic Publishers, 1995).

The term “about” includes equal to, and a range that takes into account experimental error in a given measurement and can refer to plus or minus 5, 4, 3, 2 or 1% or anywhere in-between.

As used herein, “substantially pure”, “isolated” or “purified” refers to material which is at least 50% pure (i.e., free from contaminants), more preferably at least 90% pure, more preferably at least 95% pure, more preferably at least 98% pure, more preferably at least 99% pure. Antibodies can be isolated and purified from the culture supernatant or ascites mentioned above by saturated ammonium sulfate precipitation, euglobulin precipitation method, caproic acid method, caprylic acid method, ion exchange chromatography (DEAE or DE52), or affinity chromatography using anti-Ig column or a protein A, G or L column using art-recognized conventional methods.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, .alpha.-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, the terms “hepsin”, “HPN”, or TMPRSS1” refer to a type II transmembrane serine protease (TTSP) expressed on the surface of epithelial cells. As used herein, hepsin includes all mammalian species of native sequence hepsin, e.g., human, canine, feline, equine, bovine, etc. Hepsin (HPN) is one of the most upregulated genes in human prostate cancer and encodes a type-II transmembrane serine protease that is overexpressed in up to 90% of prostate tumors with levels often increased >10 fold. Hepsin is upregulated early in prostate cancer initiation and is maintained at high levels throughout progression and metastasis. In addition, hepsin is also overexpressed in ovarian carcinomas, renal cell carcinomas (e.g., metastatic renal cell carcinomas), and in endometrial cancers. Hepsin overexpression has an important role in the promotion of prostate cancer progression and metastasis. Hepsin may activate pro-urokinase plasminogen activator (pro-uPA) and pro-hepatocyte growth factor (pro-HGF). Activation of the uPA cell-surface serine protease system and HGF-Met scattering pathway may be responsible for promotion of metastasis by hepsin. The present disclosure provides antibodies that specifically bind to clinically relevant c-terminal portion of hepsin and that may be used for diagnosis of prostate cancer, ovarian carcinomas, renal carcinomas, endometrial cancers, or a combination thereof. The 417-amino acid protein is composed of a short N-terminal cytoplasmic domain, a transmembrane domain and a single scavenger receptor cysteine-rich domain that packs tightly against the C-terminal protease domain. A native, wild-type human hepsin is provided below as SEQ ID NO: 41.

### Antibodies

As used herein, an “anti-hepsin antibody” refers to an antibody that is able to selectively bind to the c-terminal portion of hepsin. Provided herein is an isolated or a purified antibody, or antigen-binding fragment thereof, that binds to hepsin that comprises a heavy chain variable region and a light chain variable region. It would be understood that the antibodies described herein can be modified as described below or as known in the art.

“Antibodies” useful in the present invention encompass, but are not limited to, monoclonal antibodies, polyclonal antibodies, antibody fragments (e.g., Fab, Fab′, F(ab′)₂, Fv, Fc, etc.), chimeric antibodies, bispecific antibodies, multispecific antibodies, heteroconjugate antibodies, single chain (ScFv), mutants thereof, fusion proteins comprising an antibody portion (e.g., a domain antibody), humanized antibodies, human antibodies, and any other modified configuration of the immunoglobulin molecule that comprises an antigen recognition site of the required specificity, including glycosylation variants of antibodies, amino acid sequence variants of antibodies, and covalently modified antibodies.

Depending on the amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The subunit structures and three-dimensional configurations of different classes of immunoglobulins are well known in the art.

The “light chains” of antibodies (immunoglobulins) from any vertebrate species can be assigned to one of two clearly distinct types, called kappa or (“κ” or “K”) and lambda or (“λ”), based on the amino acid sequences of their constant domains.

As used herein, a “monoclonal antibody” refers to an antibody obtained from a population of substantially homogeneous antibodies, i.e., the individual antibodies comprising the population are identical except for possible naturally-occurring mutations that may be present in minor amounts. In contrast to polyclonal antibody preparations, which typically include different antibodies directed against different determinants (epitopes), each monoclonal antibody is directed against a single determinant on the antigen (epitope). The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler and Milstein, 1975, Nature, 256:495, or may be made by recombinant DNA methods such as described in U.S. Pat. No. 4,816,567. The monoclonal antibodies may also be isolated from phage libraries generated using the techniques described in McCafferty et al., 1990, Nature, 348:552-554, for example. Other methods are known in the art and are contemplated for use herein.

As used herein, “humanized” antibodies refer to forms of non-human (e.g., murine) antibodies that are specific chimeric immunoglobulins, immunoglobulin chains, or fragments thereof (such as Fv, Fab, Fab′, F(ab′)₂, scFv, or other antigen-binding subsequences of antibodies) that contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and biological activity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in, for example, WO 99/58572. Other forms of humanized antibodies have one or more CDRs (one, two, three, four, five or six) which are altered with respect to the original antibody, which are also termed one or more CDRs “derived from” one or more CDRs from the original antibody.

As used herein, a “human antibody” means an antibody having an amino acid sequence corresponding to that of an antibody produced by a human and/or has been made using any of the techniques for making human antibodies known in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nature Biotechnology, 14:309-314; Sheets et al., 1998, PNAS USA, 95:6157-6162; Hoogenboom and Winter, 1991, J. Mol. Biol., 227:381; Marks et al., 1991, J. Mol. Biol., 222:581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., 1991, J. Immunol., 147 (1):86-95; and U.S. Pat. No. 5,750,373.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al., Sequences of Proteins of Immunological Interest, (5th ed., 1991, National Institutes of Health, Bethesda Md.)); and (2) an approach based on crystallographic studies of antigen-antibody complexes (Al-Iazikani et al. (1997) J. Molec. Biol. 273:927-948)). As used herein, a CDR may refer to CDRs defined by either approach or by a combination of both approaches.

A “constant region” of an antibody refers to the constant region of the antibody light chain or the constant region of the antibody heavy chain, either alone or in combination.

“Epitope” refers to that portion of an antigen or other macromolecule capable of forming a binding interaction with the variable region binding pocket of an antibody. Such binding interactions can be manifested as an intermolecular contact with one or more amino acid residues of one or more CDRs. Antigen binding can involve, for example, a CDR3 or a CDR3 pair or, in some cases, interactions of up to all six CDRs of the V_{H} and V_{L} chains. An epitope can be a linear peptide sequence (i.e., “continuous”) or can be composed of noncontiguous amino acid sequences (i.e., “conformational” or “discontinuous”). An antibody can recognize one or more amino acid sequences; therefore, an epitope can define more than one distinct amino acid sequence. Epitopes recognized by antibodies can be determined by peptide mapping and sequence analysis techniques well known to one of skill in the art. Binding interactions are manifested as intermolecular contacts between an epitope on an antigen and one or more amino acid residues of a CDR.

An epitope that “preferentially binds” or “specifically binds” (used interchangeably herein) to an antibody or a polypeptide is a term well understood in the art, and methods to determine such specific or preferential binding are also well known in the art. An antibody specifically binds or preferentially binds to a target if it binds with greater affinity, avidity, more readily, and/or with greater duration than it binds to other substances. For example, an antibody that specifically or preferentially binds to a hepsin epitope is an antibody that binds this epitope with greater affinity, avidity, more readily, and/or with greater duration than it binds to other hepsin epitopes or non-hepsin epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding where the affinity of the antibody, or antigen-binding fragment thereof, is at least at least 2-fold greater, at least 3-fold greater, at least 4-fold greater, at least 5-fold greater, at least 6-fold greater, at least 7-fold greater, at least 8-fold greater, at least 9-fold greater, 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the antibody for unrelated amino acid sequences.

As used herein, “Fc receptor” and “FcR” describe a receptor that binds to the Fc region of an antibody.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat et al., (Sequences of Proteins of Immunological Interest, 5th Ed. Public Health Service, National Institutes of Health, Bethesda, Md., 1991). The Fc region of an immunoglobulin generally comprises two constant domains, CH2 and CH3.

A “functional Fc region” possesses at least one effector function of a native sequence Fc region. Exemplary “effector functions” include C1q binding; complement dependent cytotoxicity (CDC); Fc receptor binding; antibody-dependent cell-mediated cytotoxicity (ADCC); phagocytosis; down-regulation of cell surface receptors (e.g., B cell receptor; BCR), etc. Such effector functions generally require the Fc region to be combined with a binding domain (e.g., an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification, yet retains at least one effector function of the native sequence Fc region. Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95%, at least about 96%, at least about 97%, at least about 98%, at least about 99% sequence identity therewith.

The terms “hypervariable region” and “CDR” when used herein, refer to the amino acid residues of an antibody which are responsible for antigen-binding. The CDRs comprise amino acid residues from three sequence regions which bind in a complementary manner to an antigen and are known as CDR1, CDR2, and CDR3 for each of the V_{H} and V_{L} chains. In the light chain variable domain, the CDRs typically correspond to approximately residues 24-34 (CDRL1), 50-56 (CDRL2) and 89-97 (CDRL3), and in the heavy chain variable domain the CDRs typically correspond to approximately residues 31-35 (CDRH1), 50-65 (CDRH2) and 95-102 (CDRH3) according to Kabat et al. (Id.). It is understood that the CDRs of different antibodies may contain insertions, thus the amino acid numbering may differ. The Kabat numbering system accounts for such insertions with a numbering scheme that utilizes letters attached to specific residues (e.g., 27A, 27B, 27C, 27D, 27E, and 27F of CDRL1 in the light chain) to reflect any insertions in the numberings between different antibodies. Alternatively, in the light chain variable domain, the CDRs typically correspond to approximately residues 26-32 (CDRL1), 50-52 (CDRL2) and 91-96 (CDRL3), and in the heavy chain variable domain, the CDRs typically correspond to approximately residues 26-32 (CDRH1), 53-55 (CDRH2) and 96-101 (CDRH3) according to Chothia and Lesk, J. Mol. Biol., 196: 901-917 (1987)).

As used herein, “framework region” or “FR” refers to framework amino acid residues that form a part of the antigen binding pocket or groove. In some embodiments, the framework residues form a loop that is a part of the antigen binding pocket or groove and the amino acids residues in the loop may or may not contact the antigen. Framework regions generally comprise the regions between the CDRs. In the light chain variable domain, the FRs typically correspond to approximately residues 0-23 (FRL1), 35-49 (FRL2), 57-88 (FRL3), and 98-109 and in the heavy chain variable domain the FRs typically correspond to approximately residues 0-30 (FRH1), 36-49 (FRH2), 66-94 (FRH3), and 103-133 according to Kabat et al. (Id.). As discussed above with the Kabat numbering for the light chain, the heavy chain too accounts for insertions in a similar manner (e.g., 35A, 35B of CDRH1 in the heavy chain). Alternatively, in the light chain variable domain, the FRs typically correspond to approximately residues 0-25 (FRL1), 33-49 (FRL2) 53-90 (FRL3), and 97-109 (FRL4), and in the heavy chain variable domain, the FRs typically correspond to approximately residues 0-25 (FRH1), 33-52 (FRH2), 56-95 (FRH3), and 102-113 (FRH4) according to Chothia and Lesk (J. Mol. Biol., 196: 901-917 (1987)).

The loop amino acids of a FR can be assessed and determined by inspection of the three-dimensional structure of an antibody heavy chain and/or antibody light chain. The three-dimensional structure can be analyzed for solvent accessible amino acid positions as such positions are likely to form a loop and/or provide antigen contact in an antibody variable domain. Some of the solvent accessible positions can tolerate amino acid sequence diversity and others (e.g., structural positions) are, generally, less diversified. The three-dimensional structure of the antibody variable domain can be derived from a crystal structure or protein modeling.

As used herein, the term “affinity” refers to the equilibrium constant for the reversible binding of two agents and is expressed as K_{D}. The binding affinity (K_{D}) of an antibody described herein can be from about 0.02 pM to about 500 nM, or any integer therebetween.

Binding affinity may be determined using surface plasmon resonance (SPR), Kinexa Biocensor, scintillation proximity assays, enzyme linked immunosorbent assay (ELISA), ORIGEN immunoassay (IGEN), fluorescence quenching, fluorescence transfer, yeast display, or any combination thereof. Binding affinity may also be screened using a suitable bioassay.

As used herein, the term “avidity” refers to the resistance of a complex of two or more agents to dissociation after dilution. Apparent affinities can be determined by methods such as an enzyme linked immunosorbent assay (ELISA) or any other technique familiar to one of skill in the art. Avidities can be determined by methods such as a Scatchard analysis or any other technique familiar to one of skill in the art.

An antibody, or antigen-binding fragment thereof, can be modified by making one or more substitutions in the amino acid sequence using a conservative or a non-conservative substitution.

The phrase “conservative amino acid substitution” refers to grouping of amino acids on the basis of certain common properties. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids may be defined where amino acids within a group exchange preferentially with each other, and therefore resemble each other most in their impact on the overall protein structure. Examples of amino acid groups defined in this manner include:

a charged group, consisting of Glu and Asp, Lys, Arg and His;

(ii) a positively-charged group, consisting of Lys, Arg and His;

(iii) a negatively-charged group, consisting of Glu and Asp;

(iv) an aromatic group, consisting of Phe, Tyr and Trp;

(v) a nitrogen ring group, consisting of His and Trp;

(vi) a large aliphatic non-polar group, consisting of Val, Leu and Ile;

(vii) a slightly-polar group, consisting of Met and Cys;

(viii) a small-residue group, consisting of Ser, Thr, Asp, Asn, Gly, Ala, Glu, Gln and Pro;

(ix) an aliphatic group consisting of Val, Leu, Ile, Met and Cys; and

(x) a small hydroxyl group consisting of Ser and Thr.

In addition to the groups presented above, each amino acid residue may form its own group, and the group formed by an individual amino acid may be referred to simply by the one and/or three letter abbreviation for that amino acid commonly used in the art as described above.

A “conserved residue” is an amino acid that is relatively invariant across a range of similar proteins. Often conserved residues will vary only by being replaced with a similar amino acid, as described above for “conservative amino acid substitution.”

The letter “x” or “xaa” as used in amino acid sequences herein is intended to indicate that any of the twenty standard amino acids may be placed at this position unless specifically noted otherwise. For the purposes of peptidomimetic design, an “x” or a “xaa” in an amino acid sequence may be replaced by a mimic of the amino acid present in the target sequence, or the amino acid may be replaced by a spacer of essentially any form that does not interfere with the activity of the peptidomimetic.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present invention. In comparing two sequences, the absence of residues (amino acids or nucleic acids) or presence of extra residues also decreases the identity and homology/similarity.

The term “homology” describes a mathematically based comparison of sequence similarities which is used to identify genes or proteins with similar functions or motifs. The nucleic acid (nucleotide, oligonucleotide) and amino acid (protein) sequences of the present invention may be used as a “query sequence” to perform a search against public databases to, for example, identify other family members, related sequences or homologs. Such searches can be performed using the NBLAST and XBLAST programs (version 2.0) of Altschul, et al. (1990) J Mol. Biol. 215:403-10. BLAST nucleotide searches can be performed with the NBLAST program, score=100, wordlength=12 to obtain nucleotide sequences homologous to nucleic acid molecules of the invention. BLAST amino acid searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used (see, www.ncbi.nlm.nih.gov).

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well-known Smith Waterman algorithm may also be used to determine identity.

If needed, an antibody or an antigen binding fragment thereof described herein can be assessed for immunogenicity and, as needed, be deimmunized (i.e., the antibody is made less immunoreactive by altering one or more T cell epitopes of an antibody). Analysis of immunogenicity and T-cell epitopes present in the antibodies and antigen-binding fragments described herein can be carried out via the use of software and specific databases. Exemplary software and databases include iTope™ developed by Antitope of Cambridge, England. iTope™, which is an in-silico technology for analysis of peptide binding to human MHC class II alleles.

The iTope™ software predicts peptide binding to human MHC class II alleles and thereby provides an initial screen for the location of such “potential T cell epitopes.” iTope™ software predicts favorable interactions between amino acid side chains of a peptide and specific binding pockets within the binding grooves of 34 human MHC class II alleles. The location of key binding residues is achieved by the in-silico generation of 9mer peptides that overlap by one amino acid spanning the test antibody variable region sequence. Each 9mer peptide can be tested against each of the 34 MHC class II allotypes and scored based on their potential “fit” and interactions with the MHC class II binding groove. Peptides that produce a high mean binding score (>0.55 in the iTope™ scoring function) against >50% of the MHC class II alleles are considered as potential T cell epitopes. In such regions, the core 9 amino acid sequence for peptide binding within the MHC class II groove is analyzed to determine the MHC class II pocket residues (P1, P4, P6, P7 and P9) and the possible T cell receptor (TCR) contact residues (P-1, P2, P3, P5, P8).

After identification of any T-cell epitopes, amino acid residue changes, substitutions, additions, and/or deletions can be introduced to remove the identified T-cell epitope. Such changes can be made so as to preserve antibody structure and function while still removing the identified epitope. Exemplary changes can include, but are not limited to, conservative amino acid changes.

Provided herein are neutralizing antibodies or antigen-binding fragments that bind to hepsin and inhibit the activity of hepsin.

Percentage (%) of inhibition/neutralization by an anti-hepsin antibody or antigen-binding fragment thereof of at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, or greater than negative controls is indicative of a antibody or antigen-binding fragment thereof inhibits or neutralizes hepsin. Percentage of inhibition of hepsin by an anti-hepsin antibody or antigen-binding fragment thereof of less than 2-fold greater than negative controls is indicative of an antibody or antigen-binding fragment thereof that does not inhibit hepsin.

Antibodies, or antigen-binding fragments thereof, described herein can also be used as immunoconjugates. As used herein, for purposes of the specification and claims, immunoconjugates refer to conjugates comprised of the anti-hepsin antibodies or fragments thereof according to the present invention and at least one therapeutic label. Therapeutic labels include antitumor agents and angiogenesis-inhibitors. Such antitumor agents are known in the art and include, but not limited to, toxins, drugs, enzymes, cytokines, radionuclides, and photodynamic agents. Toxins include, but are not limited to, ricin A chain, mutant Pseudomonas exotoxins, diphtheria toxoid, streptonigrin, boamycin, saporin, gelonin, and pokeweed antiviral protein. Drugs include, but are not limited to, daunorubicin, methotrexate, and calicheamicin. Radionuclides include radiometals. Cytokines include, but are not limited to, transforming growth factor beta (TGF-β), interleukins, interferons, and tumor necrosis factors; examples of each of these cytokines and their functions are well known in the art. Photodynamic agents include, but are not limited to, porphyrins and their derivatives. Additional therapeutic labels will be known in the art and are also contemplated herein. The methods for complexing the anti-hepsin mAbs or antigen-binding fragments thereof with at least one agent are well known to those skilled in the art (i.e., antibody conjugates as reviewed by Ghetie et al., 1994, Pharmacol. Ther. 63:209-34). Such methods may utilize one of several available heterobifunctional reagents used for coupling or linking molecules. Linkers for conjugating antibodies to other moieties are well known in the art and are contemplated herein.

Methods for conjugating or linking polypeptides are well known in the art. Associations (binding) between antibodies and labels include any means known in the art including, but not limited to, covalent and non-covalent interactions, chemical conjugation as well as recombinant techniques.

Antibodies, or antigen-binding fragments thereof, can be modified using techniques known in the art for various purposes such as, for example, by addition of polyethylene glycol (PEG). PEG modification (PEGylation) can lead to one or more of improved circulation time, improved solubility, improved resistance to proteolysis, reduced antigenicity and immunogenicity, improved bioavailability, reduced toxicity, improved stability, and easier formulation (for a review, see, Francis et al., International Journal of Hematology 68:1-18, 1998).

Other methods of improving the half-life of antibody-based fusion proteins in circulation are also known such as, for example, described in U.S. Pat. Nos. 7,091,321 and 6,737,056, each of which is hereby incorporated by reference. Additionally, antibodies and antigen-binding fragments thereof may be produced or expressed so that they do not contain fucose on their complex N-glycoside-linked sugar chains. The removal of the fucose from the complex N-glycoside-linked sugar chains is known to increase effector functions of the antibodies and antigen-binding fragments, including but not limited to, antibody dependent cell-mediated cytotoxicity (ADCC) and complement dependent cytotoxicity (CDC). Similarly, antibodies or antigen-binding fragments thereof that can bind hepsin can be attached at their C-terminal end to all or part of an immunoglobulin heavy chain derived from any antibody isotype, e.g., IgG, IgA, IgE, IgD and IgM and any of the isotype sub-classes, particularly IgG1, IgG2b, IgG2a, IgG3 and IgG4.

Antibodies, or antigen-binding fragments thereof, that bind to hepsin can also be used for purification of hepsin and/or to detect hepsin levels in a sample or subject. Compositions of antibodies and antigen-binding fragments described herein can be used as non-therapeutic agents (e.g., as affinity purification agents). Generally, in one such embodiment, a protein of interest is immobilized on a solid phase such a Sephadex resin or filter paper, using conventional methods known in the art. The immobilized protein is contacted with a sample containing the target of interest (or fragment thereof) to be purified, and thereafter the support is washed with a suitable solvent that will remove substantially all the material in the sample except the target protein, which is bound to the immobilized antibody. Finally, the support is washed with another suitable solvent, such as glycine buffer, pH 5.0, which will release the target protein.

An antibody or antigen-binding fragment thereof can be conjugated to, or recombinantly engineered with, an affinity tag (e.g., a purification tag). Affinity tags such as, for example, 6x His tag (His-His-His-His-His-His; SEQ ID NO: 47) are conventional in the art.

### Methods of Making Antibodies

The antibodies described herein may be made by any method known in the art. The route and schedule of immunization of the host animal are generally in keeping with established and conventional techniques for antibody stimulation and production, as further described herein. General techniques for production of human and mouse antibodies are known in the art and are described herein and below in the Examples.

It is contemplated that any mammalian subject including humans or antibody producing cells therefrom can be manipulated to serve as the basis for production of mammalian, including human, hybridoma cell lines. Typically, the host animal is inoculated intraperitoneally, intramuscularly, orally, subcutaneously, intraplantar, and/or intradermally with an amount of immunogen, including as described herein.

Immunization of a host animal with a human protein, or a fragment containing a target amino acid sequence conjugated to an adjuvant that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaradehyde, succinic anhydride, SOCl₂, or any other adjuvant known in the art, can yield a population of antibodies.

Hybridomas can be prepared from the lymphocytes of immunized animals and immortalized myeloma cells using the general somatic cell hybridization technique of Kohler, B. and Milstein, C. (1975) Nature 256:495-497 or as modified by Buck, D. W., et al., In Vitro, 18:377-381 (1982). Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce monoclonal antibodies. The hybridomas are expanded and subcloned, if desired, and supernatants are assayed for anti-immunogen activity by conventional immunoassay procedures (e.g., radioimmunoassay, enzyme immunoassay, fluorescence immunoassay, etc.).

Hybridomas that may be used as source of antibodies encompass all derivatives, progeny cells of the parent hybridomas that produce monoclonal antibodies, or a portion thereof.

Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired.

Undesired activity, if present, can be removed, for example, by running the preparation over adsorbents made of the immunogen attached to a solid phase and eluting or releasing the desired antibodies off the immunogen.

Antibodies may be made recombinantly and expressed using any method known in the art.

Antibodies may be made recombinantly by phage display technology. See, for example, U.S. Pat. Nos. 5,565,332; 5,580,717; 5,733,743; and 6,265,150; and Winter et al., Annu. Rev. Immunol. 12:433-455 (1994). Alternatively, the phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for review see, e.g., Johnson, Kevin S, and Chiswell, David J., Current Opinion in Structural Biology, 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Mark et al., J. Mol. Biol. 222:581-597 (1991), or Griffith et al., EMBO J. 12:725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” Marks, et al., Bio/Technol. 10:779-783 (1992)). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al., Nucl. Acids Res. 21:2265-2266 (1993). Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT Publication No. WO 93/06213, published Apr. 1, 1993). Unlike traditional humanization of rodent antibodies by CDR grafting, this technique provides completely human antibodies, which have no framework or CDR residues of rodent origin.

There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; and 6,180,370.

A number of “humanized” antibody molecules comprising an antigen-binding site derived from a non-human immunoglobulin have been described, including chimeric antibodies having rodent or modified rodent V regions and their associated complementarity determining regions (CDRs) fused to human constant domains. See, for example, Winter et al. Nature 349:293-299 (1991), Lobuglio et al. Proc. Nat. Acad. Sci. USA 86:4220-4224 (1989), Shaw et al. J. Immunol. 138:4534-4538 (1987), and Brown et al. Cancer Res. 47:3577-3583 (1987).

Other references describe rodent CDRs grafted into a human supporting framework region (FR) prior to fusion with an appropriate human antibody constant domain. See, for example, Riechmann et al. Nature 332:323-327 (1988), Verhoeyen et al., Science, 239:1534-1536 (1988), and Jones et al., Nature, 321:522-525 (1986). Another reference describes rodent CDRs supported by recombinantly veneered rodent framework regions. See, for example, European Patent Publication No. 0519596. These “humanized” molecules are designed to minimize unwanted immunological response toward rodent anti-human antibody molecules which limits the duration and effectiveness of therapeutic applications of those moieties in human recipients. For example, the antibody constant region can be engineered such that it is immunologically inert (e.g., does not trigger complement lysis). See, e.g., PCT Publication No. WO 99/058572; and UK Patent Application No. 9809951.8.

Other methods of humanizing antibodies that may also be utilized are disclosed by Daugherty et al., Nucl. Acids Res., 19:2471-2476 (1991) and in U.S. Pat. Nos. 6,180,377; 6,054,297; 5,997,867; 5,866,692; 6,210,671; and 6,350,861; and in PCT Publication No. WO 01/27160.

In yet another alternative, fully human antibodies may be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are XENOMOUSE™ from Abgenix, Inc. (Fremont, Calif.) and HUMAB-MOUSE® and TC MOUSE™ from Medarex, Inc. (Princeton, N.J.).

It will be apparent that although the above discussion pertains to humanized antibodies, the general principles discussed are applicable to customizing antibodies for use, for example, in dogs, cats, primate, equines and bovines. It is further apparent that one or more aspects of humanizing an antibody described herein may be combined, e.g., CDR grafting, framework mutation and CDR mutation.

If desired, an antibody of interest may be sequenced using any known method and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity, or other characteristics of the antibody. For example, the constant region may be engineered to more resemble human constant regions to avoid immune response if the antibody is used in clinical trials and treatments in humans.

Also provided herein are methods of making any of these antibodies or polypeptides. The antibodies of this invention can be made using any conventional procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, an antibody could be produced by an automated polypeptide synthesizer employing the solid phase method. See also, U.S. Pat. Nos. 5,807,715; 4,816,567; and 6,331,415.

Antibodies may be made recombinantly by first isolating the antibodies and antibody producing cells from host animals, obtaining the gene sequence, and using the gene sequence to express the antibody recombinantly in host cells (e.g., CHO cells). Another method which may be employed is to express the antibody sequence in plants (e.g., tobacco) or transgenic milk. Methods for expressing antibodies recombinantly in plants or milk have been disclosed. See, for example, Peeters, et al. Vaccine 19:2756 (2001); Lonberg, N. and D. Huszar Int. Rev. Immunol 13:65 (1995); and Pollock, et al., J Immunol Methods 231:147 (1999). Methods for making derivatives of antibodies, e.g., single chain, etc. are known in the art.

As used herein, “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected with a polynucleotide(s) of this invention.

DNA encoding an antibody may be readily isolated and sequenced using conventional procedures (e.g., by using oligonucleotide probes that are capable of binding specifically to genes encoding the heavy and light chains of the monoclonal antibodies). Hybridoma cells may serve as a preferred source of such DNA. Once isolated, the DNA may be placed into expression vectors (such as expression vectors disclosed in PCT Publication No. WO 87/04462), which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA also may be modified, for example, by substituting the coding sequence for human heavy and light chain constant domains in place of the homologous murine sequences, Morrison et al., Proc. Nat. Acad. Sci. 81:6851 (1984), or by covalently joining to the immunoglobulin coding sequence all or part of the coding sequence for a non-immunoglobulin polypeptide. In that manner, “chimeric” or “hybrid” antibodies are prepared that have the binding specificity of an antibody described herein.

As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

In some instances, it may be desirable to genetically manipulate an antibody sequence to obtain greater affinity to hepsin and greater efficacy in inhibiting and/or neutralizing hepsin. It will be apparent to one of skill in the art that one or more polynucleotide changes can be made to the antibody and still maintain its binding ability to hepsin.

An expression vector can be used to direct expression of an antibody. One skilled in the art is familiar with administration of expression vectors to obtain expression of an exogenous protein in vivo. See, e.g., U.S. Pat. Nos. 6,436,908; 6,413,942; and 6,376,471.

Single chain variable region fragments (“scFv”) of antibodies are described herein. Single chain variable region fragments may be made by linking light and/or heavy chain variable regions by using a short linking peptide. Bird et al. (1988) Science 242:423-426. An example of a linking peptide is (GGGGS)3 (SEQ ID NO: 49) which bridges approximately 3.5 nm between the carboxy terminus of one variable region and the amino terminus of the other variable region. Linkers of other sequences have been designed and used. Bird et al. (Id). Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al., Proc. Natl. Acad. Sci. USA, 90:6444-6448 (1993); and Poljak, R. J., et al., Structure, 2:1121-1123 (1994)).

For example, bispecific antibodies, monoclonal antibodies that have binding specificities for at least two different antigens, can be prepared using the antibodies disclosed herein. Methods for making bispecific antibodies are known in the art (see, e.g., Suresh et al., 1986, Methods in Enzymology 121:210). Traditionally, the recombinant production of bispecific antibodies was based on the coexpression of two immunoglobulin heavy chain-light chain pairs, with the two heavy chains having different specificities (Millstein and Cuello, 1983, Nature, 305, 537-539). Bispecific antibodies can be composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations. This approach is described in PCT Publication No. WO 94/04690.

According to one approach to making bispecific antibodies, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1), containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

Heteroconjugate antibodies, comprising two covalently joined antibodies, are also within the scope of the invention. Such antibodies have been used to target immune system cells to unwanted cells (U.S. Pat. No. 4,676,980). Heteroconjugate antibodies may be made using any convenient cross-linking methods. Suitable cross-linking agents and techniques are well known in the art such as described in U.S. Pat. No. 4,676,980.

Chimeric or hybrid antibodies also may be prepared in vitro using known methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

Anti-hepsin antibodies, and antigen-binding fragments thereof, can be identified or characterized using methods known in the art, whereby reduction, amelioration, or neutralization of a hepsin biological activity is detected and/or measured.

Antibodies may be characterized using methods well known in the art. For example, one method is to identify the epitope to which it binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an anti-hepsin antibody binds. Epitope mapping is commercially available from various sources, for example, Pepscan Systems (Edelhertweg 15, 8219 PH Lelystad, The Netherlands). The epitope can be a linear epitope, i.e., contained in a single stretch of amino acids, or a conformational epitope formed by a three-dimensional interaction of amino acids that may not necessarily be contained in a single stretch. Peptides of varying lengths (e.g., at least 4-6 amino acids long) can be isolated or synthesized (e.g., recombinantly) and used for binding assays with an anti-hepsin antibody. In another example, the epitope to which the anti-hepsin antibody binds can be determined in a systematic screening by using overlapping peptides derived from the anti-hepsin sequence and determining binding by the anti-hepsin antibody. According to the gene fragment expression assays, the open reading frame encoding hepsin is fragmented either randomly or by specific genetic constructions and the reactivity of the expressed fragments of hepsin with the antibody to be tested is determined. The gene fragments may, for example, be produced by PCR and then transcribed and translated into protein in vitro, in the presence of radioactive amino acids. The binding of the antibody to the radioactively labeled hepsin fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant hepsin in which various fragments of the hepsin polypeptide have been replaced (swapped) with sequences from a closely related, but antigenically distinct protein. By assessing binding of the antibody to the mutant hepsin, the importance of the particular hepsin fragment to antibody binding can be assessed.

Yet another method which can be used to characterize an anti-hepsin antibody is to use competition assays with other antibodies known to bind to the same antigen, i.e., various fragments on hepsin, to determine if the anti-hepsin antibody binds to the same epitope as other antibodies. Competition assays are well known to those of skill in the art.

Also provided herein are affinity matured antibodies. For example, affinity matured antibodies can be produced by procedures known in the art (Marks et al., 1992, Bio/Technology, 10:779-783; Barbas et al., 1994, Proc Nat. Acad. Sci, USA 91:3809-3813; Schier et al., 1995, Gene, 169:147-155; Yelton et al., 1995, J. Immunol., 155:1994-2004; Jackson et al., 1995, J. Immunol., 154(7):3310-9; Hawkins et al, 1992, J. Mol. Biol., 226:889-896; and WO2004/058184).

The following methods may be used for adjusting the affinity of an antibody and for characterizing a CDR. One way of characterizing a CDR of an antibody and/or altering (such as improving) the binding affinity of a polypeptide, such as an antibody, termed “library scanning mutagenesis”. Generally, library scanning mutagenesis works as follows. One or more amino acid positions in the CDR are replaced with two or more (such as 3, 4, 5, 6, 7, 8, 9, 10, 11, 12, 13, 14, 15, 16, 17, 18, 19, or 20) amino acids using art recognized methods. This generates small libraries of clones (in some embodiments, one for every amino acid position that is analyzed), each with a complexity of two or more members (if two or more amino acids are substituted at every position). Generally, the library also includes a clone comprising the native (unsubstituted) amino acid. A small number of clones, e.g., about 20-80 clones (depending on the complexity of the library), from each library are screened for binding affinity to the target polypeptide (or other binding target), and candidates with increased, the same, decreased or no binding are identified. Methods for determining binding affinity are well-known in the art. Binding affinity may be determined using Biacore surface plasmon resonance analysis, which detects differences in binding affinity of about 2-fold or greater. Biacore is particularly useful when the starting antibody already binds with a relatively high affinity, for example a K_{D} of about 10 nM or lower.

In some embodiments, every amino acid position in a CDR is replaced (in some embodiments, one at a time) with all 20 natural amino acids using art recognized mutagenesis methods (some of which are described herein). This generates small libraries of clones (in some embodiments, one for every amino acid position that is analyzed), each with a complexity of 20 members (if all 20 amino acids are substituted at every position).

In some embodiments, the library to be screened comprises substitutions in two or more positions, which may be in the same CDR or in two or more CDRs. Thus, the library may comprise substitutions in two or more positions in one CDR. The library may comprise substitution in two or more positions in two or more CDRs. The library may comprise substitution in 3, 4, 5, or more positions, said positions found in two, three, four, five or six CDRs. The substitution may be prepared using low redundancy codons. See, e.g., Table 2 of Balint et al., Gene, 137(1):109-18 (1993). The CDR may be CDRH3 and/or CDRL3. The CDR may be one or more of CDRL1, CDRL2, CDRL3, CDRH1, CDRH2, and/or CDRH3. The CDR may be a Kabat CDR, a Chothia CDR, or an extended CDR.

Candidates with improved binding may be sequenced, thereby identifying a CDR substitution mutant which results in improved affinity (also termed an “improved” substitution). Candidates that bind may also be sequenced, thereby identifying a CDR substitution which retains binding.

Multiple rounds of screening may be conducted. For example, candidates (each comprising an amino acid substitution at one or more position of one or more CDR) with improved binding are also useful for the design of a second library containing at least the original and substituted amino acid at each improved CDR position (i.e., amino acid position in the CDR at which a substitution mutant showed improved binding). Preparation, and screening or selection of this library is discussed further below.

Library scanning mutagenesis also provides a means for characterizing a CDR, in so far as the frequency of clones with improved binding, the same binding, decreased binding or no binding also provide information relating to the importance of each amino acid position for the stability of the antibody-antigen complex. For example, if a position of the CDR retains binding when changed to all 20 amino acids, that position is identified as a position that is unlikely to be required for antigen binding. Conversely, if a position of CDR retains binding in only a small percentage of substitutions, that position is identified as a position that is important to CDR function. Thus, the library scanning mutagenesis methods generate information regarding positions in the CDRs that can be changed to many different amino acids (including all 20 amino acids), and positions in the CDRs which cannot be changed or which can only be changed to a few amino acids.

Candidates with improved affinity may be combined in a second library, which includes the improved amino acid, the original amino acid at that position, and may further include additional substitutions at that position, depending on the complexity of the library that is desired, or permitted using the desired screening or selection method. In addition, if desired, adjacent amino acid position can be randomized to at least two or more amino acids. Randomization of adjacent amino acids may permit additional conformational flexibility in the mutant CDR, which may in turn, permit or facilitate the introduction of a larger number of improving mutations. The library may also comprise substitution at positions that did not show improved affinity in the first round of screening.

The second library is screened or selected for library members with improved and/or altered binding affinity using any method known in the art, including screening using Biacore surface plasmon resonance analysis, and selection using any method known in the art for selection, including phage display, yeast display, and ribosome display.

Exemplary Anti-Hepsin Antibody Amino Acid Sequences

In one aspect, the present disclosure provides for an isolated antibody that selectively binds to circulating hepsin or to the c-terminus of circulating hepsin. An anti-hepsin antibody described herein, in some instances, does not selectively bind to the serine proteases Matripase, KLK6, KLK7, and KLK8. In the exemplary VH and VL sequences provided below, it would be understood that the signal sequence is not to be included in the final variable region sequences.

In one aspect, an antibody or antigen-binding fragment thereof comprises a VH sequence having at least 90%, 91%, 92%, 93%, 94%, 95%, 96%, 97%, 98%, 99%, or 100% sequence identity to the amino acid sequence of any one of SEQ ID NOS: 1, 2, and 3. In some embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody comprising that sequence retains the ability to bind to same antigen as of the parent (e.g., cancer associated antigen). In some embodiments, a total of 1 to 10 amino acids have been substituted, inserted and/or deleted in the amino acid sequence of any one of SEQ ID NOS: 1, 2, and 3. In some embodiments, substitutions, insertions, or deletions occur in regions outside the CDRs (e.g., in the FRs). Optionally, the antibody comprises the VH sequence of the amino acid sequence of any one of SEQ ID NOS: 1, 2, and 3, including one or more post-translational modifications of that sequence. In a particular embodiment, the VH comprises one, two or three CDRs selected from: (a) CDR-H1 comprising the amino acid sequence of any one of SEQ ID NOS: 16 (KYWMS), 18 (SGYSWH), and 19 (SFGMH), (b) CDR-H2 comprising the amino acid sequence of any one of SEQ ID NOS: 11 (EINPDGSTIIYTPSLKD), 13 (YIHYNGNTNYNPSLKS), and 14 (YISSGSSAIYYADTVKG), and (c) CDR-H3 comprising the amino acid sequence of any one of SEQ ID NOS: 6 (RANWYYFDY), (ADF), and 9 (SNWDYFDY). In certain embodiments, the amino acids of the CDR-H1, CDR-H2, and/or CDR-H3 are defined by Chothia numbering. In certain embodiments, the amino acids of the CDR-H1, CDR-H2, and/or CDR-H3 are defined by Martin numbering. In certain embodiments, the amino acids of the CDR-H1, CDR-H2, and/or CDR-H3 are defined by Kabat numbering. In certain embodiments, the amino acids of the CDR-H1, CDR-H2, and/or CDR-H3 are defined by AHo numbering. In certain embodiments, the amino acids of the CDR-H1, CDR-H2, and/or CDR-H3 are defined by IMGT numbering.

In one aspect, an antibody or antigen-binding fragment thereof, is provided, wherein the antibody or antigen-binding fragment thereof comprises a light chain variable domain (VL) having at least 90%, 91%, 92%, 93%, 94%, 95%, 96%, 97%, 98%, 99%, or 100% sequence identity to the amino acid sequence of SEQ ID NO: 4 or 5. In some embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to same antigen as the parent (e.g., cancer associated antigen). In some embodiments, a total of 1 to 10 amino acids have been substituted, inserted and/or deleted in the amino acid sequence of SEQ ID NO: 4 or 5. In some embodiments, the substitutions, insertions, or deletions occur in regions outside the CDRs (e.g., in the FRs). Optionally, the antibody or antigen-binding fragment thereof comprises the VL sequence of SEQ ID NO: 4 or 5, including post-translational modifications of that sequence. In a particular embodiment, the VL comprises one, two or three CDRs selected from (a) CDR-L1 comprising the amino acid sequence of SEQ ID NO: 17 (TASSSVSSSNFH) or 20; (b) CDR-L2 comprising the amino acid sequence of SEQ ID NO: 12 (STSNLAS) or 15; and (c) CDR-L3 comprising the amino acid sequence of SEQ ID NO: 7 (HQYHRSPRT) or 10. In certain embodiments, the amino acids of the CDR-L1, CDR-L2, and/or CDR-L3 are defined by Chothia numbering. In certain embodiments, the amino acids of the CDR-L1, CDR-L2, and/or CDR-L3 are defined by Martin numbering. In certain embodiments, the amino acids of the CDR-L1, CDR-L2, and/or CDR-L3 are defined by Kabat numbering. In certain embodiments, the amino acids of the CDR-L1, CDR-L2, and/or CDR-L3 are defined by AHo numbering. In certain embodiments, the amino acids of the CDR-L1, CDR-L2, and/or CDR-L3 are defined by IMGT numbering.

In one aspect, an antibody or antigen-binding fragment thereof is provided, wherein the antibody or antigen-binding fragment thereof comprises a VH as in any of the embodiments provided above, and a VL as in any of the embodiments provided above. In some embodiments, the antibody or antigen-binding fragment thereof comprises a VH and a VL, wherein the VH comprises the amino acid sequence of any one of SEQ ID NOS: 1, 2, and 3, and wherein the VL comprises the amino acid sequence in SEQ ID NO: 4 or 5, and optionally including post-translational modifications of those sequences.

In one aspect, an antibody or antigen-binding fragment thereof is provided, wherein the antibody or antigen-binding fragment thereof comprises a VH selected from any VH in Table 1. In one aspect, an antibody or antigen-binding fragment thereof is provided, wherein the antibody or antigen-binding fragment thereof comprises a VL selected from any VL in Table 1. In one aspect, an antibody or antigen-binding fragment thereof is provided, wherein the antibody or antigen-binding fragment thereof comprises a VH selected from any VH in Table 1 and a VL selected from any VL in Table 1. In one aspect, an antibody or antigen-binding fragment thereof is provided, wherein the antibody or antigen-binding fragment thereof comprises a VH selected from any VH in Table 1 and a VL selected from any VL in Table 1, wherein the selected VH and VL are paired according to Table 5. In one aspect, an antibody or antigen-binding fragment thereof is provided, wherein the antibody or antigen-binding fragment thereof comprises a CDR-H3 selected from any CDR-H3 in Table 2 and a CDR-L3 selected from any CDRL3 in Table 2, wherein the selected CDR-H3 and CDRL3 are paired according to Table 5. In one aspect, an antibody or antigen-binding fragment thereof is provided, wherein the antibody or antigen-binding fragment thereof comprises a CDR-H2 selected from any CDR-H2 in Table 2 and a CDR-L2 selected from any CDR-L2 in Table 2, wherein the selected CDR-H2 and CDR-L2 are paired according to Table 5. In one aspect, an antibody or antigen-binding fragment thereof is provided, wherein the antibody or antigen-binding fragment thereof comprises a CDR-H1 selected from any CDR-H1 in Table 2 and a CDR-L1 selected from any CDR-L1 in Table 2, wherein the selected CDR-H1 and CDR-L1 are paired according to Table 5. In one aspect, an antibody or antigen-binding fragment thereof is provided, wherein the antibody or antigen-binding fragment thereof comprises a CDR-H1, a CDR-H2, and a CDR-H3 selected from any CDR-H1, a CDR-H2, and a CDR-H3 in Table 2 and a CDR-L1, a CDR-L2, and a CDR-L3 selected from any CDR-L1, CDR-L2, or CDR-L3 in Table 2, wherein the selected CDR-H1, CDR-H2, CDR-H3, CDR-L1, CDR-L2, and CDR-L3 are paired according to Table 5. In certain embodiments, the amino acids of the CDR-H1, CDR-H2, and/or CDR-H3 are defined by Chothia numbering. In certain embodiments, the amino acids of the CDR-H1, CDR-H2, and/or CDR-H3 are defined by Martin numbering. In certain embodiments, the amino acids of the CDR-H1, CDR-H2, and/or CDR-H3 are defined by Kabat numbering. In certain embodiments, the amino acids of the CDR-H1, CDR-H2, and/or CDR-H3 are defined by AHo numbering. In certain embodiments, the amino acids of the CDR-H1, CDR-H2, and/or CDR-H3 are defined by IMGT numbering. In certain embodiments, the amino acids of the CDR-L1, CDR-L2, and/or CDR-L3 are defined by Chothia numbering. In certain embodiments, the amino acids of the CDR-L1, CDR-L2, and/or CDR-L3 are defined by Martin numbering. In certain embodiments, the amino acids of the CDR-L1, CDR-L2, and/or CDR-L3 are defined by Kabat numbering. In certain embodiments, the amino acids of the CDR-L1, CDR-L2, and/or CDR-L3 are defined by AHo numbering. In certain embodiments, the amino acids of the CDR-L1, CDR-L2, and/or CDR-L3 are defined by IMGT numbering.

H2a

In one aspect, the present disclosure provides an antibody or antigen-binding fragment thereof comprises one or more variable regions selected from the group consisting of (a) VH comprising the amino acid sequence of SEQ ID NO: 1, (b) VL comprising the amino acid sequence of SEQ ID NO: 4, and (c) a combination thereof.

In one aspect, the present disclosure provides an antibody or antigen-binding fragment thereof comprising at least one, two, three, four, five, or six CDRs selected from (a) CDR-H1 comprising the amino acid sequence of SEQ ID NO: 16 (KYWMS); (b) CDR-H2 comprising the amino acid sequence of SEQ ID NO: 11 (EINPDGSTIIYTPSLKD); (c) CDR-H3 comprising the amino acid sequence of SEQ ID NO: 6 (RANWYYFDY); (d) CDR-L1 comprising the amino acid sequence of SEQ ID NO: 17 (TASSSVSSSNFH); (e) CDR-L2 comprising the amino acid sequence of SEQ ID NO: 12 (STSNLAS); and (f) CDR-L3 comprising the amino acid sequence of SEQ ID NO:7.

In one aspect, the present disclosure provides an antibody or antigen-binding fragment thereof comprising at least one, at least two, or all three VH CDR sequences selected from (a) CDR-H1 comprising the amino acid sequence of SEQ ID NO: 16 (KYWMS); (b) CDR-H2 comprising the amino acid sequence of SEQ ID NO: 11 (EINPDGSTIIYTPSLKD); and (c) CDR-H3 comprising the amino acid sequence of SEQ ID NO: 6 (RANWYYFDY); and (d) a VL comprising the amino acid sequence of SEQ ID NO: 4.

In one aspect, the present disclosure provides an antibody or antigen-binding fragment thereof comprising at least one, at least two, or all three VL CDR sequences selected from (a) CDR-L1 comprising the amino acid sequence of SEQ ID NO: 17 (TASSSVSSSNFH); (b) CDR-L2 comprising the amino acid sequence of SEQ ID NO: 12 (STSNLAS); and (c) CDR-L3 comprising the amino acid sequence of SEQ ID NO: 7 (HQYHRSPRT); and a VH comprising the amino acid sequence of SEQ ID NO: 1.

In one aspect, the present disclosure provides an antibody or antigen-binding fragment thereof comprising the CDRs: CDR-H3 comprising the amino acid sequence of SEQ ID NO: 6 (RANWYYFDY); and CDR-L3 comprising the amino acid sequence of SEQ ID NO: 7 (HQYHRSPRT).

In one aspect, the disclosure herein provides an antibody or antigen-binding fragment thereof comprising at least one, at least two, or all three VL CDR sequences selected from (a) CDR-L1 comprising the amino acid sequence of SEQ ID NO: 17 (TASSSVSSSNFH); (b) CDR-L2 comprising the amino acid sequence of SEQ ID NO: 12 (STSNLAS); and (c) CDR-L3 comprising the amino acid sequence of SEQ ID NO: 7 (HQYHRSPRT). In one aspect, the disclosure provides an antibody or antigen-binding fragment thereof comprising at least one, at least two, or all three VH CDR sequences selected from (a) CDR-H1 comprising the amino acid sequence of SEQ ID NO: 16 (KYWMS); (b) CDR-H2 comprising the amino acid sequence of SEQ ID NO: 11 (EINPDGSTIIYTPSLKD); and (c) CDR-H3 comprising the amino acid sequence of SEQ ID NO: 6 (RANWYYFDY).

In one aspect, the disclosure provides an antibody or antigen-binding fragment thereof comprising the CDRs: (a) CDR-H1 comprising the amino acid sequence of SEQ ID NO: 16 (KYWMS); (b) CDR-H2 comprising the amino acid sequence of SEQ ID NO: 11 (EINPDGSTIIYTPSLKD); (c) CDR-H3 comprising the amino acid sequence of SEQ ID NO: 6 (RANWYYFDY); (d) CDR-L1 comprising the amino acid sequence of SEQ ID NO: 17 (TASSSVSSSNFH); (e) CDR-L2 comprising the amino acid sequence of SEQ ID NO: 12 (STSNLAS); and (f) CDR-L3 comprising the amino acid sequence of SEQ ID NO: 7 (HQYHRSPRT).

In one aspect, an antibody or antigen-binding fragment thereof comprises a VH sequence having at least 90%, 91%, 92%, 93%, 94%, 95%, 96%, 97%, 98%, 99%, or 100% sequence identity to the amino acid sequence of SEQ ID NO: 1. In some embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in the amino acid sequence of SEQ ID NO: 1. In some embodiments, substitutions, insertions, or deletions occur in regions outside the CDRs (e.g., in the FRs). Optionally, the antibody or antigen-binding fragment thereof comprises the VH sequence of the amino acid sequence of SEQ ID NO: 1, including post-translational modifications of that sequence. In a particular embodiment, the VH comprises one, two or three CDRs selected from: (a) CDR-H1 comprising the amino acid sequence of SEQ ID NO: 16 (KYWMS), (b) CDR-H2 comprising the amino acid sequence of SEQ ID NO: 11 (EINPDGSTIIYTPSLKD), and (c) CDR-H3 comprising the amino acid sequence of SEQ ID NO: 6 (RANWYYFDY).

In one aspect, an antibody or antigen-binding fragment thereof is provided, wherein the antibody or antigen-binding fragment thereof comprises a light chain variable domain (VL) having at least 90%, 91%, 92%, 93%, 94%, 95%, 96%, 97%, 98%, 99%, or 100% sequence identity to the amino acid sequence of SEQ ID NO: 4. In some embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in the amino acid sequence of SEQ ID NO: 4. In some embodiments, the substitutions, insertions, or deletions occur in regions outside the CDRs (e.g., in the FRs). Optionally, the antibody or antigen-binding fragment thereof comprises the VL sequence of SEQ ID NO: 4, including post-translational modifications of that sequence. In a particular embodiment, the VL comprises one, two or three CDRs selected from (a) CDR-L1 comprising the amino acid sequence of SEQ ID NO: 17 (TASSSVSSSNFH); (b) CDR-L2 comprising the amino acid sequence of SEQ ID NO: 12 (STSNLAS); and (c) CDR-L3 comprising the amino acid sequence of SEQ ID NO: 7 (HQYHRSPRT).

In one aspect, an antibody or antigen-binding fragment thereof is provided, wherein the antibody or antigen-binding fragment thereof comprises a VH as in any of the embodiments provided above, and a VL as in any of the embodiments provided above. In some embodiments, the antibody comprises a VH comprising the amino acid sequence of SEQ ID NO: 1, and a VL sequence in SEQ ID NO: 4, including post-translational modifications of those sequences.

H2b

In one aspect, the present disclosure provides an antibody or antigen-binding fragment thereof comprises one or more variable regions selected from the group consisting of (a) VH comprising the amino acid sequence of SEQ ID NO: 2, (b) VL comprising the amino acid sequence of SEQ ID NO: 4, and (c) a combination thereof.

In one aspect, the present disclosure provides an antibody or antigen-binding fragment thereof comprising at least one, two, three, four, five, or six CDRs selected from (a) CDR-H1 comprising the amino acid sequence of SEQ ID NO: 18 (SGYSWH); (b) CDR-H2 comprising the amino acid sequence of SEQ ID NO: 13 (YIHYNGNTNYNPSLKS); (c) CDR-H3 comprising the amino acid sequence of (ADF); (d) CDR-L1 comprising the amino acid sequence of SEQ ID NO: 17 (TASSSVSSSNFH); (e) CDR-L2 comprising the amino acid sequence of SEQ ID NO: 12 (STSNLAS); and (f) CDR-L3 comprising the amino acid sequence of SEQ ID NO:7.

In one aspect, the present disclosure provides an antibody or antigen-binding fragment thereof comprising at least one, at least two, or all three VH CDR sequences selected from (a) CDR-H1 comprising the amino acid sequence of SEQ ID NO: 18 (SGYSWH); (b) CDR-H2 comprising the amino acid sequence of SEQ ID NO: 13 (YIHYNGNTNYNPSLKS); and (c) CDR-H3 comprising the amino acid sequence of (ADF); and (d) a VL comprising the amino acid sequence of SEQ ID NO: 4.

In one aspect, the present disclosure provides an antibody or antigen-binding fragment thereof comprising at least one, at least two, or all three VL CDR sequences selected from (a) CDR-L1 comprising the amino acid sequence of SEQ ID NO: 17 (TASSSVSSSNFH); (b) CDR-L2 comprising the amino acid sequence of SEQ ID NO: 12 (STSNLAS); and (c) CDR-L3 comprising the amino acid sequence of SEQ ID NO: 7 (HQYHRSPRT); and a VH comprising the amino acid sequence of SEQ ID NO: 2.

In one aspect, the present disclosure provides an antibody or antigen-binding fragment thereof comprising the CDRs: CDR-H3 comprising the amino acid sequence of (ADF); and CDR-L3 comprising the amino acid sequence of SEQ ID NO: 7 (HQYHRSPRT).

In one aspect, the disclosure herein provides an antibody or antigen-binding fragment thereof comprising at least one, at least two, or all three VL CDR sequences selected from (a) CDR-L1 comprising the amino acid sequence of SEQ ID NO: 17 (TASSSVSSSNFH); (b) CDR-L2 comprising the amino acid sequence of SEQ ID NO: 12 (STSNLAS); and (c) CDR-L3 comprising the amino acid sequence of SEQ ID NO: 7 (HQYHRSPRT). In one aspect, the disclosure provides an antibody or antigen-binding fragment thereof comprising at least one, at least two, or all three VH CDR sequences selected from (a) CDR-H1 comprising the amino acid sequence of SEQ ID NO: 18 (SGYSWH); (b) CDR-H2 comprising the amino acid sequence of SEQ ID NO: 13 (YIHYNGNTNYNPSLKS); and (c) CDR-H3 comprising the amino acid sequence of (ADF).

In one aspect, the disclosure provides an antibody or antigen-binding fragment thereof comprising the CDRs: (a) CDR-H1 comprising the amino acid sequence of SEQ ID NO: 18 (SGYSWH); (b) CDR-H2 comprising the amino acid sequence of SEQ ID NO: 13 (YIHYNGNTNYNPSLKS); (c) CDR-H3 comprising the amino acid sequence of (ADF); (d) CDR-L1 comprising the amino acid sequence of SEQ ID NO: 17 (TASSSVSSSNFH); (e) CDR-L2 comprising the amino acid sequence of SEQ ID NO: 12 (STSNLAS); and (f) CDR-L3 comprising the amino acid sequence of SEQ ID NO: 7 (HQYHRSPRT).

In one aspect, an antibody or antigen-binding fragment thereof comprises a VH sequence having at least 90%, 91%, 92%, 93%, 94%, 95%, 96%, 97%, 98%, 99%, or 100% sequence identity to the amino acid sequence of SEQ ID NO: 2. In some embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in the amino acid sequence of SEQ ID NO: 2. In some embodiments, substitutions, insertions, or deletions occur in regions outside the CDRs (e.g., in the FRs). Optionally, the antibody or antigen-binding fragment thereof comprises the VH sequence of the amino acid sequence of SEQ ID NO: 2, including post-translational modifications of that sequence. In a particular embodiment, the VH comprises one, two or three CDRs selected from: (a) CDR-H1 comprising the amino acid sequence of SEQ ID NO: 18 (SGYSWH), (b) CDR-H2 comprising the amino acid sequence of SEQ ID NO: 13 (YIHYNGNTNYNPSLKS), and (c) CDR-H3 comprising the amino acid sequence of (ADF).

In one aspect, an antibody or antigen-binding fragment thereof is provided, wherein the antibody or antigen-binding fragment thereof comprises a light chain variable domain (VL) having at least 90%, 91%, 92%, 93%, 94%, 95%, 96%, 97%, 98%, 99%, or 100% sequence identity to the amino acid sequence of SEQ ID NO: 4. In some embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in the amino acid sequence of SEQ ID NO: 4. In some embodiments, the substitutions, insertions, or deletions occur in regions outside the CDRs (e.g., in the FRs). Optionally, the antibody or antigen-binding fragment thereof comprises the VL sequence of SEQ ID NO: 4, including post-translational modifications of that sequence. In a particular embodiment, the VL comprises one, two or three CDRs selected from (a) CDR-L1 comprising the amino acid sequence of SEQ ID NO: 17 (TASSSVSSSNFH); (b) CDR-L2 comprising the amino acid sequence of SEQ ID NO: 12 (STSNLAS); and (c) CDR-L3 comprising the amino acid sequence of SEQ ID NO: 7 (HQYHRSPRT).

In one aspect, an antibody or antigen-binding fragment thereof is provided, wherein the antibody or antigen-binding fragment thereof comprises a VH as in any of the embodiments provided above, and a VL as in any of the embodiments provided above. In some embodiments, the antibody comprises a VH comprising the amino acid sequence of SEQ ID NO: 2, and a VL sequence in SEQ ID NO: 4, including post-translational modifications of those sequences.

H5

In one aspect, the present disclosure provides an antibody or antigen-binding fragment thereof comprising at least one or both variable regions selected from (a) VH comprising the amino acid sequence of SEQ ID NO: 3 and (b) VL comprising the amino acid sequence of SEQ ID NO: 5.

In one aspect, the present disclosure provides an antibody or antigen-binding fragment thereof comprising at least one, two, three, four, five, or six CDRs selected from (a) CDR-H1 comprising the amino acid sequence of SEQ ID NO: 19 (SFGMH); (b) CDR-H2 comprising the amino acid sequence of SEQ ID NO: 14 (YISSGSSAIYYADTVKG); (c) CDR-H3 comprising the amino acid sequence of SEQ ID NO: 9 (SNWDYFDY); (d) CDR-L1 comprising the amino acid sequence of SEQ ID NO: 20 (KSSQSLLNSRIRKNYLA); (e) CDR-L2 comprising the amino acid sequence of SEQ ID NO: 15 (WASTRES); and (f) CDR-L3 comprising the amino acid sequence of SEQ ID NO: 10 (KQSYNLWT).

In one aspect, the present disclosure provides an antibody or antigen-binding fragment thereof comprising at least one, at least two, or all three VH CDR sequences selected from (a) CDR-H1 comprising the amino acid sequence of SEQ ID NO: 19 (SFGMH); (b) CDR-H2 comprising the amino acid sequence of SEQ ID NO: 14 (YISSGSSAIYYADTVKG); and (c) CDR-H3 comprising the amino acid sequence of SEQ ID NO: 9 (SNWDYFDY); and (d) a VL comprising the amino acid sequence of SEQ ID NO: 5.

In one aspect, the present disclosure provides an antibody or antigen-binding fragment thereof comprising at least one, at least two, or all three VL CDR sequences selected from (a) CDR-L1 comprising the amino acid sequence of SEQ ID NO: 20 (KSSQSLLNSRIRKNYLA); (b) CDR-L2 comprising the amino acid sequence of SEQ ID NO: 15 (WASTRES); and (c) CDR-L3 comprising the amino acid sequence of SEQ ID NO: 10 (KQSYNLWT); and a VH comprising the amino acid sequence of SEQ ID NO: 3.

In one aspect, the present disclosure provides an antibody or antigen-binding fragment thereof comprising the CDRs: CDR-H3 comprising the amino acid sequence of SEQ ID NO: 9 (SNWDYFDY); and CDR-L3 comprising the amino acid sequence of SEQ ID NO: 10 (KQSYNLWT).

In one aspect, the disclosure herein provides an antibody or antigen-binding fragment thereof comprising at least one, at least two, or all three VL CDR sequences selected from (a) CDR-L1 comprising the amino acid sequence of SEQ ID NO: 20 (KSSQSLLNSRIRKNYLA); (b) CDR-L2 comprising the amino acid sequence of SEQ ID NO: 15 (WASTRES); and (c) CDR-L3 comprising the amino acid sequence of SEQ ID NO: 10 (KQSYNLWT). In one aspect, the disclosure provides an antibody or antigen-binding fragment thereof comprising at least one, at least two, or all three VH CDR sequences selected from (a) CDR-H1 comprising the amino acid sequence of SEQ ID NO: 19 (SFGMH); (b) CDR-H2 comprising the amino acid sequence of SEQ ID NO: 14 (YISSGSSAIYYADTVKG); and (c) CDR-H3 comprising the amino acid sequence of SEQ ID NO: 9 (SNWDYFDY).

In one aspect, the disclosure provides an antibody or antigen-binding fragment thereof comprising the CDRs: (a) CDR-H1 comprising the amino acid sequence of SEQ ID NO: 19 (SFGMH); (b) CDR-H2 comprising the amino acid sequence of SEQ ID NO: 14 (YISSGSSAIYYADTVKG); (c) CDR-H3 comprising the amino acid sequence of SEQ ID NO: 9 (SNWDYFDY); (d) CDR-L1 comprising the amino acid sequence of SEQ ID NO: 20 (KSSQSLLNSRIRKNYLA); (e) CDR-L2 comprising the amino acid sequence of SEQ ID NO: 15 (WASTRES); and (f) CDR-L3 comprising the amino acid sequence of SEQ ID NO: 10 (KQSYNLWT).

In one aspect, an antibody or antigen-binding fragment thereof comprises a VH sequence having at least 90%, 91%, 92%, 93%, 94%, 95%, 96%, 97%, 98%, 99%, or 100% sequence identity to the amino acid sequence of SEQ ID NO: 3. In some embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in the amino acid sequence of SEQ ID NO: 3. In some embodiments, substitutions, insertions, or deletions occur in regions outside the CDRs (e.g., in the FRs). Optionally, the antibody or antigen-binding fragment thereof comprises the VH sequence of the amino acid sequence of SEQ ID NO: 3, including post-translational modifications of that sequence. In a particular embodiment, the VH comprises one, two or three CDRs selected from: (a) CDR-H1 comprising the amino acid sequence of SEQ ID NO: 19 (SFGMH), (b) CDR-H2 comprising the amino acid sequence of SEQ ID NO: 14 (YISSGSSAIYYADTVKG), and (c) CDR-H3 comprising the amino acid sequence of SEQ ID NO: 9 (SNWDYFDY).

In one aspect, an antibody or antigen-binding fragment thereof is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 5. In some embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in the amino acid sequence of SEQ ID NO: 5. In some embodiments, the substitutions, insertions, or deletions occur in regions outside the CDRs (e.g., in the FRs). Optionally, the antibody or antigen-binding fragment thereof comprises the VL sequence of SEQ ID NO: 5, including post-translational modifications of that sequence. In a particular embodiment, the VL comprises one, two or three CDRs selected from (a) CDR-L1 comprising the amino acid sequence of SEQ ID NO: 20 (KSSQSLLNSRIRKNYLA); (b) CDR-L2 comprising the amino acid sequence of SEQ ID NO: 15 (WASTRES); and (c) CDR-L3 comprising the amino acid sequence of SEQ ID NO: 10 (KQSYNLWT).

In one aspect, an antibody or antigen-binding fragment thereof is provided, wherein the antibody or antigen-binding fragment thereof comprises a VH as in any of the embodiments provided above, and a VL as in any of the embodiments provided above. In some embodiments, the antibody or antigen-binding fragment thereof comprises a VH comprising the amino acid sequence of SEQ ID NO: 3, and a VL sequence in SEQ ID NO: 5, including post-translational modifications of those sequences.

Exemplary Nucleic Acid Sequences

The disclosure provides isolated nucleic acid comprising a sequence encoding an antibody polypeptide or antigen-binding fragment thereof. In some embodiments, the isolated nucleic acid comprises a sequence encoding a heavy chain polypeptide of an antibody. See, Tables 3, 4, and 6. In some embodiments, the nucleic acid sequence encoding a heavy chain polypeptide is selected from any one of SEQ ID NOS: 21, 22, and 23. In some embodiments, the isolated nucleic acid comprises a sequence encoding a light chain polypeptide of an antibody. In some embodiments, the nucleic acid sequence encoding a light chain polypeptide is selected from any one of SEQ ID NOS: 24 or 25. In some embodiments, the isolated nucleic acid comprises a sequence encoding a CDR1 polypeptide of a variable heavy chain. In some embodiments, the isolated nucleic acid comprises a sequence encoding a CDR2 polypeptide of a variable heavy chain. In some embodiments, the isolated nucleic acid comprises a sequence encoding a CDR3 polypeptide of a variable heavy chain. In some embodiments, the nucleic acid sequence encoding the CDR1 polypeptide of a variable heavy chain is selected from any one of SEQ ID NOS: 16 (KYWMS), 18 (SGYSWH), and 19 (SFGMH). In some embodiments, the nucleic acid sequence encoding the CDR2 polypeptide of a variable heavy chain is selected from any one of SEQ ID NOS: 11 (EINPDGSTIIYTPSLKD), 13 (YIHYNGNTNYNPSLKS), and 14 (YISSGSSAIYYADTVKG). In some embodiments, the nucleic acid sequence encoding the CDR3 polypeptide of a variable heavy chain is selected from any one of SEQ ID NOS: 6 (RANWYYFDY), (ADF), and 9 (SNWDYFDY). In some embodiments, the isolated nucleic acid comprises a sequence encoding a CDR1 polypeptide of a variable light chain. In some embodiments, the isolated nucleic acid comprises a sequence encoding a CDR2 polypeptide of a variable light chain. In some embodiments, the isolated nucleic acid comprises a sequence encoding a CDR3 polypeptide of a variable light chain. In some, embodiments, the nucleic acid sequence encoding the CDR1 region of a variable light chain polypeptide is selected from any one of SEQ ID NOS: 17 (TASSSVSSSNFH) and 20 (KSSQSLLNSRIRKNYLA). In some, embodiments, the nucleic acid sequence encoding the CDR2 region of a variable light chain polypeptide is selected from any one of SEQ ID NOS: 12 (STSNLAS) and 15 (WASTRES). In some, embodiments, the nucleic acid sequence encoding the CDR3 region of a variable light chain polypeptide is selected from any one of SEQ ID NOS: 7 (HQYHRSPRT) and 10 (KQSYNLWT).

Exemplary Methods of Producing Antibodies

In another aspect, present disclosure provides for a method of producing an antibody that selectively binds to circulating hepsin, the method comprising screening hybridomas generated by immunizing an animal to hepsin against an isolated c-terminal portion of hepsin.

In some instances, the antibody is preparable by the method of: (a) preparing hybridomas (e.g., from a rodent immunized with a recombinant hepsin sequence that lacks a transmembrane portion (e.g., of wild-type hepsin)); (b) screening the hybridomas of a) against serum obtained from an individual (e.g., diagnosed with (e.g., an epithelial) cancer); and (c) isolating hybridomas of b) that specifically bind to circulating (or extracellular) hepsin.

In some instances, the antibody is preparable by the method of: (a) preparing hybridomas (e.g., from a rodent immunized with a recombinant hepsin sequence that lacks a transmembrane portion (e.g., of wild-type hepsin)); (b) screening the hybridomas of b) against serum obtained from an individual (e.g., diagnosed with (e.g., an epithelial) cancer); (c) isolating hybridomas of b) that specifically bind to extracellular hepsin; (d) screening the hybridomas of c) against a recombinant, biologically-active extracellular hepsin; and (e) isolating the hybridomas of d) that specifically bind to the extracellular, circulating c-terminal portion of hepsin.

The hepsin utilized to prepare the antibodies described herein, in some instances, comprises human hepsin. In some instances of such methods, the recombinant, biologically-active extracellular hepsin further comprises a thrombin cleavage site and, optionally, a spacer. The circulating hepsin of such methods, in some instances, comprises an amino acid sequence of SEQ ID NO: 44. The wild-type human hepsin utilized in such methods, in some cases, comprises an amino acid sequence of SEQ ID NO: 41.

In another aspect, present disclosure provides for an isolated antibody that binds to a recombinant hepsin sequence that comprises an amino acid sequence of SEQ ID NO: 43. In some instances, the antibody, wherein the binding is selective.

In another aspect, present disclosure provides for a composition that comprises an antibody of any one of the preceding claims and a pharmaceutically acceptable excipient.

### Compositions and Kits

An antibody described herein may be prepared as a lyophilized form or an aqueous solution for storage. A lyophilized antibody may be reconstituted for use by mixing the antibody having the desired degree of purity with one or more acceptable carriers, excipients, or stabilizers. Acceptable carriers, excipients, or stabilizers are generally nontoxic and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosacchandes, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). One or more of such agents may extend the half-life of the antibody in storage.

Provided herein are compositions of antibodies and antigen-binding fragments thereof that bind hepsin and include those such as described elsewhere herein. Antibodies and antigen-binding fragments thereof that bind hepsin as described herein can be used for the diagnosis of various forms cancer described below.

Also provided herein are kits for use in the instant methods. Kits may include one or more containers comprising an anti-hepsin antibody described herein and instructions for use in accordance with any of the methods described herein. Generally, these instructions comprise a description of administration of the anti-hepsin antibody to diagnose a cancer according to any of the methods described herein.

The containers may be single units or multi units of containers containing an antibody described herein. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the antibody is used for diagnosing a cancer such as, for example, a prostate cancer, an ovarian cancer, or a carcinoma. In one instance, the cancer is an ovarian cancer. In another instance, the cancer is a prostate cancer. In another instance, the cancer is a carcinoma. Non-limiting examples of carcinomas include, but are not limited to, a renal cell carcinoma (RCC) or a metastatic renal cell carcinoma. Instructions may be provided for practicing any of the methods described herein.

The kits may be provided in suitable packaging. Suitable packaging includes, but is not limited to, vials, bottles, jars, flexible packaging (e.g., sealed Mylar or plastic bags), and the like. Kits may optionally provide additional components such as buffers for reconstitution of a lyophilized antibody and interpretive information. Normally, the kit comprises a container and a label or package insert(s) on or associated with the container.

### Methods of Diagnosis

One aspect of the present disclosure relates to diagnosis of a disorder associated with elevated levels of hepsin in a subject. An “individual” or a “subject” to be diagnosed by a method herein may be a mammal, more preferably a human. Mammals also include, but are not limited to, farm animals, sport animals, and pets, including, but not limited to, primates, equines, bovines, alpacas, dogs, cats, rabbits, mice and rats.

In one instance, the disorder associated with elevated levels of hepsin is a cancer or a tumor. The terms “cancer” and “tumor” are used herein to refer to a cancerous tissue (as compared to expression by normal tissue of the same type). Tumors can include solid tumors (cancers) and semi-solid tumors (cancers). Tumors (cancers) may also, in some instances, be metastatic. In one instance, a tumor or a cancer is an epithelial cancer. Examples of tumors (cancers) include, but are not limited to, a prostate cancer, an ovarian cancer, or a combination thereof. Other examples of epithelial tumors (cancers) include, but are not limited to, a carcinoma, an adenocarcinoma, or a combination thereof. In one instance, the cancer is an ovarian cancer. In another instance, the cancer is a prostate cancer. In another instance, the cancer is a carcinoma. Non-limiting examples of carcinomas include, but are not limited to, a renal cell carcinoma (RCC) or a metastatic renal cell carcinoma.

Assessment may be performed based on objective measures such as, for example, testing a biological sample obtained from a subject. Further types of assessments are described below. Assessment may also be performed based on subjective measures, such as characterization of symptoms of a subject.

One aspect of the present disclosure provides for a method of identifying the presence of circulating hepsin in a biological sample, the method comprising: (a) contacting the biological sample (e.g., obtained from an individual, such as an individual suspected of having or at risk for cancer) with an antibody that selectively binds circulating hepsin (e.g., the antibodies described herein); and (b) determining whether circulating hepsin is present in the biological sample (e.g., determining whether the amount of circulating hepsin is elevated. In some embodiments, the antibody that selectively binds to circulating hepsin comprises (1) a variable heavy chain CDR-H1, CDR-H2 and CDR-H3, wherein CDR-H1 comprises a reconstructed polypeptide consensus sequence selected from any one of SEQ ID NOS: 16 (KYWMS), 18 (SGYSWH), and 19 (SFGMH), CDR-H2 comprises a reconstructed polypeptide consensus sequence selected from any one of SEQ ID NOS: 11 (EINPDGSTIIYTPSLKD), 13 (YIHYNGNTNYNPSLKS), and 14 (YISSGSSAIYYADTVKG), and CDR-H3 comprises a reconstructed polypeptide consensus sequence selected from any one of SEQ ID NOS: 6 (RANWYYFDY), (ADF), and 9 (SNWDYFDY); and/or (2) a variable light chain CDR-L1, CDR-L2 and CDR-L3, wherein CDR-L1 comprises a reconstructed polypeptide consensus sequence selected from any one of SEQ ID NOS: 17 (TASSSVSSSNFH) and 20 (KSSQSLLNSRIRKNYLA), CDR-L2 comprises a reconstructed polypeptide consensus sequence selected from any one of SEQ ID NOS: 12 (STSNLAS) and 15 (WASTRES), and CDR-L3 comprises a reconstructed polypeptide consensus sequence selected from any one of SEQ ID NOS: 7 (HQYHRSPRT) and 10 (KQSYNLWT). In some embodiments, the antibody that selectively binds to circulating hepsin comprises (1) a variable heavy chain CDR-H1, CDR-H2 and CDR-H3 selected from any one of SEQ ID NOS: 1-3, wherein the CDR-H1, CDR-H2, and CDR-H3 are defined by Chothia numbering, Martin numbering, Kabat numbering, AHo numbering, or IMGT numbering; and/or (2) a variable light chain CDR-L1, CDR-L2 and CDR-L3 selected from any one of SEQ ID NOS: 4-5, wherein the CDR-L1, CDR-L2, and CDR-L3 are defined by Chothia numbering, Martin numbering, Kabat numbering, AHo numbering, or IMGT numbering.

A “biological sample” as used herein, includes, but is not limited to, any quantity of a substance from a living thing or formerly living thing such as, for example, humans, mice, rats, monkeys, dogs, rabbits, and other animals. Such samples include, but are not limited to, blood (or non-tissue) sample (e.g., whole blood, serum, urine, etc.), cells, organs, tissues, and combinations thereof. In some instances, a biological sample is treated or modified prior to use in a diagnostic method described herein. For example, heparin may be added to a blood sample and serum collected. If a sample is a tissue sample, fluid around the tissue may be collected for use, or a tissue sample may be homogenized in a buffered solution prior to use in a diagnostic method described herein.

Any suitable assay may be utilized for the described diagnostic methods including, but not limited to, an enzyme-linked immunosorbent assay (ELISA), ELISPOT, immunohistochemistry (IHC), antibody adaptation to microbeads for multiplex and/or microfluidic platforms.

In an aspect, detectably labeled antibodies that bind circulating hepsin are useful for the detection of a circulating hepsin in a sample. Accordingly, in some embodiments, the antibody that binds circulating hepsin detectable moiety is selected from the group consisting of a fluorescent label, an enzyme, a colloidal metal, a magnetic particle, and a latex bead. In certain

In an aspect, the detection of circulating hepsin can be achieved using a lateral flow assay device that provides for point-of-care analysis. In certain instances, the use of a lateral flow assay provides for the effective detection of circulating hepsin in a sample. In certain instances, a lateral flow assay device refers to any device that receives fluid, such as a sample, and includes a laterally disposed fluid transport or flow path along which various sites or zones are provided for supporting various reagents (e.g., antibodies that bind circulating hepsin) through which sample traverses under the influence of capillary forces or other applied forces, and in which lateral flow assays are conducted for the detection of at least one analyte of interest. For example, in some embodiments, a sample is loaded onto a membrane having a first zone comprising a detectable antibody that binds circulating hepsin and a second zone for the detection of the detectable antibody bound to circulating hepsin, wherein capillary forces bring the sample into contact with the detectable antibodies of the first zone and the second zone. If a sample comprises circulating hepsin, the detectable antibodies of the first zone will bind the circulating hepsin and be detected in the second zone. Accordingly, further provided herein are lateral flow assay device or compositions comprising an antibody that binds circulating hepsin, as described herein. Also provided are lateral flow assay device or compositions for use in a method of detecting circulating hepsin, wherein the lateral flow assay device or compositions comprise an antibody that binds circulating hepsin. In certain embodiments, the method comprises, contacting an antibody that binds to circulating hepsin with a sample, and detecting the presence of the antibody bound to circulating hepsin. In certain instances, the advantages of the antibodies that bind circulating hepsin described herein enable the effective detection of circulating hepsin in a sample via a lateral flow assay device or composition. In some embodiments the lateral flow assay device or composition is configured to provide a limit of deletion of greater than 50 ng/mL, 100 ng/mL, 200 ng/mL, 300 ng/mL, 500 ng/mL or greater.

In some of such diagnostic methods, the method further comprises identifying presence of or risk of developing cancer in a subject (e.g., the subject suspected of or at risk for cancer). In some of such diagnostic methods, the method further comprises identifying risk of recurrence of cancer in a subject (e.g., a subject suspected of being at risk for recurrence). In some of such diagnostic methods, the method further comprises identifying risk of metastasis of cancer in an individual (e.g., an individual suspected of being at risk for recurrence).

A cancer to be detected using the diagnostic methods described herein comprises an epithelial cancer. Epithelial cancers to be diagnoses using the methods described herein include, but are not limited to, an ovarian cancer, a prostate cancer, a carcinoma, or a combination thereof. In one instance, the cancer is an ovarian cancer. In another instance, the cancer is a prostate cancer. In another instance, the cancer is a carcinoma. Non-limiting examples of carcinomas include, but are not limited to, a renal cell carcinoma (RCC) or a metastatic renal cell carcinoma.

### Methods of Treatment

One aspect of the present disclosure relates to the treatment of a disorder associated with elevated levels of circulating hepsin. An “individual” or a “subject” to be treated by a method herein may be a mammal, more preferably a human. Mammals also include, but are not limited to, farm animals, sport animals, and pets, including, but not limited to, primates, equines, bovines, alpacas, dogs, cats, rabbits, mice, and rats. It will be appreciated that a “subject in need thereof” may be suffering from a disorder associated with elevated levels of circulating hepsin.

As used herein, a “therapeutically effective dosage” or a “therapeutically effective amount” of a pharmaceutical composition described herein is an amount sufficient to result in beneficial or desired results in a subject. Beneficial or desired results include results such as lessening the severity or delaying the progression of the disorder, including biochemical, histological and/or behavioral symptoms of the disorder, its complications and intermediate pathological phenotypes presenting during development of the disorder.

As is understood in the clinical context, an effective dosage of a pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved. Accordingly, in some instances, one or more therapeutic agents may be administered to the subject. In other instances, treatment with a pharmaceutical composition described herein is conducted prior to, or after, one or more other treatment modalities described herein.

Various formulations of an anti-hepsin antibody may be used for administration. In some embodiments, the anti-hepsin antibody may be administered neat. In some embodiments, the anti-hepsin antibody and a pharmaceutically acceptable excipient may be in various formulations. Pharmaceutically acceptable excipients are known in the art and include relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and non-parenteral drug delivery are set forth in Remington, The Science and Practice of Pharmacy 20th Ed. Mack Publishing (2000).

The antibodies may be formulated for administration by any suitable means. The particular dosage regimen, i.e., dose, timing and repetition, will depend on the particular individual and that individual's medical history. In some instances, these antibodies are formulated for administration by injection (e.g., intravenously, intraperitoneally, subcutaneously, intramuscularly, etc.). Accordingly, these antibodies can be combined with pharmaceutically acceptable vehicles such as saline, Ringer's solution, dextrose solution, and the like. In other instances, the antibodies can also be administered via inhalation.

Generally, for administration of anti-hepsin antibodies, an initial dosage can be about 2 mg/kg. For the purpose of the present invention, a typical daily dosage can be up to 3 μg/kg, up to about 30 μg/kg, up to about 300 μg/kg, up to about 3 mg/kg, up to 30 mg/kg, up to 100 mg/kg or more, or any integer therebetween, depending on the factors mentioned above. For example, a dosage of about 1 mg/kg, about 2.5 mg/kg, about 5 mg/kg, about 10 mg/kg, or about 25 mg/kg may be used. For repeated administrations over several days or longer, depending on the condition, the treatment is sustained until a desired suppression of symptoms occurs or until sufficient therapeutic levels are achieved, for example, to reduce pain. An exemplary dosing regimen comprises administering an initial dose of about 2 mg/kg, followed by a weekly maintenance dose of about 1 mg/kg of the anti-hepsin antibody, or followed by a maintenance dose of about 1 mg/kg every other week.

However, other dosage regimens may be useful, depending on the pattern of pharmacokinetic decay that the practitioner wishes to achieve. For example, in some embodiments, dosing from one-four times a week is contemplated. The progress of this therapy is easily monitored by conventional techniques and assays. The dosing regimen can vary over time. The appropriate dosage of an anti-hepsin antibody will depend on the anti-hepsin antibody employed, the type and stage of cancer to be treated, previous surgery and/or therapy, the subject's clinical history and response to the antibody, and the discretion of the attending physician. Typically, a clinician will administer an anti-hepsin antibody, until a dosage is reached that achieves the desired result. Dose and/or frequency can vary over course of treatment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system.

Frequency of administration may be determined and adjusted over the course of therapy. Alternatively, sustained continuous release formulations of anti-hepsin antibodies may be appropriate. Various formulations and devices for achieving sustained release are known in the art. To assess efficacy of an anti-hepsin antibody, an indicator of the disease can be followed.

Treatment includes, for example, about 2-fold, about 3-fold, about 4-fold, about 5-fold, about 10-fold, about 20-fold, about 50-fold, or any fold reduction in between of one or more symptoms. Similarly, treatment can include about 1%, about 2%, about 3%, about 4%, about 5%, about 10%, about 20%, about 30%, about 40%, about 50%, about 60%, about 70%, about 80%, about 90%, about 95%, about 99%, 100%, or any percentage reduction in between or one or more symptoms.

One aspect of the present disclosure relates to a method of treating a disorder associated with elevated levels of circulating hepsin in a subject in need thereof, comprising administering to the subject an antibody that selectively binds to circulating hepsin as described herein.

Another aspect of the present disclosure relates to a method of treating hyperhepsinemia in a subject in need thereof, comprising administering to the subject an antibody that selectively binds to circulating hepsin as described herein. In some embodiments, the antibody that selectively binds to circulating hepsin comprises (1) a variable heavy chain CDR-H1, CDR-H2 and CDR-H3, wherein CDR-H1 comprises a reconstructed polypeptide consensus sequence selected from any one of SEQ ID NOS: 16 (KYWMS), 18 (SGYSWH), and 19 (SFGMH), CDR-H2 comprises a reconstructed polypeptide consensus sequence selected from any one of SEQ ID NOS: 11 (EINPDGSTIIYTPSLKD), 13 (YIHYNGNTNYNPSLKS), and 14 (YISSGSSAIYYADTVKG), and CDR-H3 comprises a reconstructed polypeptide consensus sequence selected from any one of SEQ ID NOS: 6 (RANWYYFDY), (ADF), and 9 (SNWDYFDY); and/or (2) a variable light chain CDR-L1, CDR-L2 and CDR-L3, wherein CDR-L1 comprises a reconstructed polypeptide consensus sequence selected from any one of SEQ ID NOS: 17 (TASSSVSSSNFH) and 20 (KSSQSLLNSRIRKNYLA), CDR-L2 comprises a reconstructed polypeptide consensus sequence selected from any one of SEQ ID NOS: 12 (STSNLAS) and 15 (WASTRES), and CDR-L3 comprises a reconstructed polypeptide consensus sequence selected from any one of SEQ ID NOS: 7 (HQYHRSPRT) and 10 (KQSYNLWT). In some embodiments, the antibody that selectively binds to circulating hepsin comprises (1) a variable heavy chain CDR-H1, CDR-H2 and CDR-H3 selected from any one of SEQ ID NOS: 1-3, wherein the CDR-H1, CDR-H2, and CDR-H3 are defined by Chothia numbering, Martin numbering, Kabat numbering, AHo numbering, or IMGT numbering; and/or (2) a variable light chain CDR-L1, CDR-L2 and CDR-L3 selected from any one of SEQ ID NOS: 4-5, wherein the CDR-L1, CDR-L2, and CDR-L3 are defined by Chothia numbering, Martin numbering, Kabat numbering, AHo numbering, or IMGT numbering.

Treatment is continued until one or more symptoms of the disorders are partially or completely resolved.

### Exemplary Sequences

Exemplary amino acid and nucleic acid sequences of variable heavy chains, variable light chains, and their corresponding CDR are provided below.

TABLE 1
lists exemplary amino acid sequences of variable
heavy chain (VH) and exemplary amino acid
sequences variable light chain (VL).

| | | SEQ | |
|---|---|---|---|
| | | ID | |
| Name | Chain | No | Amino acid sequences |
| H2 | VH1 | 1 | Signal peptide-FR1-CDR1-FR2-CDR2- |
| | | | FR3-CDR3-FR4 |
| | | | MDFGLIFFIVALLKGVQCEVKLLESGGGLVQPGG |
| | | | SLKLSCAASGFDFNKYWMSWVRQAPGKGLEWIGE |
| | | | INPDGSTIIYTPSLKDKFIISRDNARNTLYLQMS |
| | | | KVRSEDTALYYCARRANWYYFDYWGQGTTLTVSS |
| | VL | 4 | Signal peptide-FR1-CDR1-FR2-CDR2- |
| | | | FR3-CDR3-FR4 |
| | | | MDFQVQIFSFLLISASVIMSRGQIVLTQSPAIMS |
| | | | ASLGERVTMTCTASSSVSSSNFHWYQQKPGSSPK |
| | | | LWIYSTSNLASGVPTRFSGSGSGTSYSLTISSME |
| | | | AEDAATYYCHQYHRSPRTFGGGTKLEIK |
| | VH2 | 2 | Signal peptide-FR1-CDR1-FR2-CDR2- |
| | | | FR3-CDR3-FR4 |
| | | | MRVLILLCLFTAFPGILSDVQLQESGPDLVKPSQ |
| | | | SLSLTCTVTGYSITSGYSWHWIRQFPGNILEWMA |
| | | | YIHYNGNTNYNPSLKSRISITRDTSKNQFFLQLN |
| | | | SVTTEDTATYFCARADFWGQGTTLTVSS |
| H5 | VH | 3 | Signal peptide-FR1-CDR1-FR2-CDR2- |
| | | | FR3-CDR3-FR4 |
| | | | MDSRLNLVFLVLILKGVQCDVQLVESGGDLVQPG |
| | | | ESRKLSCAASGFTFSSFGMHWVRQAPEKGLEWVA |
| | | | YISSGSSAIYYADTVKGRFTISRDNPKNTLFLHM |
| | | | TSLRSEDTAIYYCAGSNWDYFDYWGQGTTLTVSS |
| | VL | 5 | Signal peptide-FR1-CDR1-FR2-CDR2- |
| | | | FR3-CDR3-FR4 |
| | | | MDSQAQVLILLLLWVSGTCEDIVMSQSPSSLAVS |
| | | | AGEKVTMSCKSSQSLLNSRIRKNYLAWYQQKPGQ |
| | | | SPKLLIYWASTRESGVPDRFTGSGSGTDFTLTIS |
| | | | SVQAEDLAVYYCKQSYNLWTFGGGTKLEIK |

Table 2 below lists exemplary amino acid sequences of complementarity determining regions from variable heavy chains (CDR-H) and exemplary amino acid sequences of complementarity determining regions from variable light chains (CDR-L).

| | Region | SEQ | Amino acid sequences of |
|---|---|---|---|
| | and | ID | complementarity |
| Name | Chain | No | determining regions |
| H2 | VH1 CDR-H3 | 6 | RANWYYFDY |
| | CDR-L3 | 7 | HQYHRSPRT |
| | VH2 CDR-H3 | | ADF |
| H5 | CDR-H3 | 9 | SNWDYFDY |
| | CDR-L3 | 10 | KQSYNLWT |
| H2 | VH1 CDR-H2 | 11 | EINPDGSTIIYTPSLKD |
| | CDR-L2 | 12 | STSNLAS |
| | VH2 CDR-H2 | 13 | YIHYNGNTNYNPSLKS |
| H5 | CDR-H2 | 14 | YISSGSSAIYYADTVKG |
| | CDR-L2 | 15 | WASTRES |
| H2 | VH1 CDR-H1 | 16 | KYWMS |
| | CDR-L1 | 17 | TASSSVSSSNFH |
| | VH2 CDR-H1 | 18 | SGYSWH |
| H5 | CDR-H1 | 19 | SFGMH |
| | CDR-L1 | 20 | KSSQSLLNSRIRKNYLA |

Table 3 below lists exemplary nucleic acid sequences of variable heavy chains (VH) and exemplary nucleic acid sequences of variable light chains (VL).

| | | SEQ | |
|---|---|---|---|
| | | ID | |
| Name | Chain | No | Nucleic acid sequences |
| H2 | VH | 21 | Signal sequence-FR1-CDR1-FR2-CDR2- |
| | | | FR3-CDR3-FR4 |
| | | | ATGGATTTTGGGCTGATTTTTTTTATTGTTGCTC |
| | | | TTTTAAAAGGGGTCCAGTGTGAGGTGAAACTTCT |
| | | | CGAGTCTGGAGGTGGCCTGGTGCAGCCTGGAGGA |
| | | | TCCCTGAAACTCTCCTGTGCAGCCTCAGGATTCG |
| | | | ATTTTAATAAATACTGGATGAGTTGGGTCCGGCA |
| | | | GGCTCCAGGGAAAGGGCTAGAATGGATTGGAGAA |
| | | | ATTAATCCAGATGGCAGTACGATCATCTATACGC |
| | | | CATCTCTAAAGGATAAATTCATCATCTCCAGAGA |
| | | | CAACGCCAGAAATACTCTGTACCTGCAAATGAGC |
| | | | AAAGTGAGATCTGAGGACACAGCCCTTTATTACT |
| | | | GTGCAAGACGGGCTAACTGGTACTACTTTGACTA |
| | | | CTGGGGCCAAGGCACCACTCTCACAGTCTCCTCA |
| | VL | 24 | Signal sequence-FR1-CDR1-FR2-CDR2- |
| | | | FR3-CDR3-FR4 |
| | | | ATGGATTTTCAGGTGCAGATTTTCAGCTTCCTGC |
| | | | TAATCAGTGCCTCAGTCATAATGTCCAGAGGACA |
| | | | AATTGTTCTCACCCAGTCTCCAGCAATCATGTCT |
| | | | GCATCTCTAGGGGAACGGGTCACCATGACCTGCA |
| | | | CTGCCAGCTCAAGTGTAAGTTCCAGTAACTTTCA |
| | | | CTGGTACCAGCAGAAGCCAGGATCCTCCCCCAAA |
| | | | CTCTGGATTTATAGCACCTCCAACCTGGCTTCTG |
| | | | GAGTCCCAACTCGCTTCAGTGGCAGTGGGTCTGG |
| | | | GACCTCTTACTCTCTCACAATCAGCAGCATGGAG |
| | | | GCTGAAGATGCTGCCACTTATTACTGCCACCAGT |
| | | | ATCATCGTTCCCCACGGACGTTCGGTGGAGGCAC |
| | | | CAAGCTGGAAATCAAA |
| | VH2 | 22 | Signal sequence-FR1-CDR1-FR2-CDR2- |
| | | | FR3-CDR3-FR4 |
| | | | ATGAGAGTGCTGATTCTTTTGTGCCTGTTCACAG |
| | | | CCTTTCCTGGTATCCTGTCTGATGTGCAGCTTCA |
| | | | GGAGTCAGGACCTGACCTGGTGAAACCTTCTCAG |
| | | | TCACTTTCACTCACCTGCACTGTCACTGGCTACT |
| | | | CCATCACCAGTGGTTATAGCTGGCACTGGATCCG |
| | | | GCAGTTTCCAGGAAACATACTGGAATGGATGGCC |
| | | | TACATACACTACAATGGTAACACTAACTACAACC |
| | | | CATCTCTCAAAAGTCGGATCTCTATCACTCGAGA |
| | | | CACCTCCAAGAACCAGTTCTTCCTGCAGTTGAAT |
| | | | TCTGTGACTACTGAGGACACTGCCACATATTTCT |
| | | | GTGCAAGAGCCGATTTCTGGGGCCAAGGCACCAC |
| | | | TCTCACAGTCTCCTCA |
| H5 | VH | 23 | Signal sequence-FR1-CDR1-FR2-CDR2- |
| | | | FR3-CDR3-FR4 |
| | | | ATGGACTCCAGGCTCAATTTAGTTTTCCTTGTCC |
| | | | TTATTTTAAAAGGTGTCCAGTGTGATGTGCAGCT |
| | | | GGTGGAGTCTGGGGGAGACTTAGTGCAGCCTGGA |
| | | | GAGTCCCGGAAACTCTCCTGTGCAGCCTCTGGAT |
| | | | TCACTTTCAGTAGCTTTGGAATGCACTGGGTTCG |
| | | | TCAGGCTCCAGAGAAGGGGCTGGAGTGGGTCGCA |
| | | | TACATTAGTAGTGGCAGTAGTGCCATCTACTATG |
| | | | CAGACACAGTGAAGGGCCGATTCACCATCTCCAG |
| | | | AGACAATCCCAAGAACACCCTGTTCCTACACATG |
| | | | ACCAGTCTAAGGTCTGAGGACACGGCCATATATT |
| | | | ACTGTGCAGGAAGCAACTGGGACTACTTTGACTA |
| | | | CTGGGGCCAAGGCACCACTCTCACAGTCTCCTCA |
| | VL | 25 | Signal sequence-FR1-CDR1-FR2-CDR2- |
| | | | FR3-CDR3-FR4 |
| | | | ATGGATTCACAGGCCCAGGTTCTTATATTGCTGC |
| | | | TGCTATGGGTATCTGGTACCTGTGAGGACATTGT |
| | | | GATGTCACAGTCTCCATCCTCCCTGGCTGTGTCA |
| | | | GCAGGAGAGAAGGTCACTATGAGCTGCAAATCCA |
| | | | GTCAGAGTCTGCTAAACAGTAGAATCCGAAAGAA |
| | | | CTACTTGGCTTGGTACCAGCAGAAACCAGGGCAG |
| | | | TCTCCTAAATTGTTGATCTACTGGGCATCCACTA |
| | | | GGGAATCTGGGGTCCCTGATCGCTTCACAGGCAG |
| | | | TGGATCTGGGACAGATTTCACTCTCACCATCAGC |
| | | | AGTGTGCAGGCTGAGGACCTGGCAGTTTATTACT |
| | | | GCAAACAATCTTATAATCTGTGGACGTTCGGTGG |
| | | | AGGCACCAAGCTGGAAATCAAA |

Table 4 below lists exemplary nucleic acid sequences of complementarity determining region from variable heavy chains (CDR-H) and exemplary nucleic acid sequences of complementarity determining region from variable light chains (CDR-L). The start and stop position on the corresponding isolated nucleic acid sequence is indicated.

| | Region | SEQ | Start | Stop | Nucleic acid sequences |
|---|---|---|---|---|---|
| | and | ID | posi- | posi- | of complementarity |
| Name | Chain | No | tion | tion | determining regions |
| H2 | VH1 | 26 | 289 | 351 | CGGGCTAACTGGTACTACTTTGA |
| | CDR- | | | | CTAC |
| | H3 | | | | |
| | CDR- | 29 | 270 | 299 | CACCAGTATCATCGTTCCCCACG |
| | L3 | | | | GACG |
| | VH2 | | 289 | 324 | GCCGATTTC |
| | CDR- | | | | |
| | H3 | | | | |
| H5 | CDR- | 28 | 289 | 327 | AGCAACTGGGACTACTTTGACTA |
| | H3 | | | | C |
| | CDR- | 30 | 270 | 299 | AAACAATCTTATAATCTGTGGAC |
| | L3 | | | | G |
| H2 | VH1 | 31 | 151 | 174 | GAAATTAATCCAGATGGCAGTAC |
| | CDR- | | | | GATCATCTATACGCCATCTCTAA |
| | H2 | | | | AGGAT |
| | CDR- | 34 | 153 | 161 | AGCACCTCCAACCTGGCTTCT |
| | L2 | | | | |
| | VH2 | 32 | 151 | 174 | TACATACACTACAATGGTAACAC |
| | CDR- | | | | TAACTACAACCCATCTCTCAAAA |
| | H2 | | | | GT |
| H5 | CDR- | 33 | 151 | 174 | TACATTAGTAGTGGCAGTAGTGC |
| | H2 | | | | CAATCTACTTGCAGACACAGTGA |
| | | | | | AGGGC |
| | CDR- | 35 | 153 | 161 | AAACAATCTTATAATCTGTGGAC |
| | L2 | | | | G |
| H2 | VH1 | 36 | 76 | 99 | AAATACTGGATGAGT |
| | CDR- | | | | |
| | H1 | | | | |
| | CDR- | 39 | 81 | 101 | ACTGCCAGCTCAAGTGTAAGTTC |
| | L1 | | | | CAGTAACTTTCAC |
| | VH2 | 37 | 76 | 99 | AGTGGTTATAGCTGGCAC |
| | CDR- | | | | |
| | H1 | | | | |
| H5 | CDR- | 38 | 76 | 99 | AGCTTTGGAATGCAC |
| | H1 | | | | |
| | CDR- | 40 | 81 | 101 | AAATCCAGTCAGAGTCTGCTAAA |
| | L1 | | | | CAGTAGAATCCGAAAGAACTACT |
| | | | | | TGGCT |

Table 5 lists exemplary amino acid sequence heavy and light chain pairings.

| | SEQ ID Nos | | | | | | | |
|---|---|---|---|---|---|---|---|---|
| | Variable | | CDR3 | | CDR2 | | CDR1 | |
| | Region | | Heavy | Light | Heavy | Light | Heavy | Light |
| | Heavy | Light | Chain | chain | Chain | chain | Chain | chain |
| | Chain | Chain | (CDR- | (CDR- | (CDR- | (CDR- | (CDR- | (CDR- |
| Name | (VH) | (VL) | H3) | L3) | H2) | L2) | H1) | L1) |
| H2a | 1 | 4 | 6 | 7 | 11 | 12 | 16 | 17 |
| H2b | 2 | 4 | 8 | 7 | 13 | 12 | 18 | 17 |
| H5 | 3 | 5 | 9 | 10 | 14 | 15 | 19 | 20 |

Table 6 lists exemplary nucleic acid sequence heavy and light chain pairings.

| | SEQ ID Nos | | | | | | | |
|---|---|---|---|---|---|---|---|---|
| | Variable | | CDR3 | | CDR2 | | CDR1 | |
| | Region | | Heavy | Light | Heavy | Light | Heavy | Light |
| | Heavy | Light | Chain | chain | Chain | chain | Chain | chain |
| | Chain | Chain | (CDR- | (CDR- | (CDR- | (CDR- | (CDR- | (CDR- |
| Name | (VH) | (VL) | H3) | L3) | H2) | L2) | H1) | L1) |
| H2a | 21 | 24 | 26 | 29 | 31 | 34 | 36 | 39 |
| H2b | 22 | 24 | 27 | 29 | 32 | 34 | 37 | 39 |
| H5 | 23 | 25 | 28 | 30 | 33 | 35 | 38 | 40 |

```
SEQ ID NO: 41: native, wild-type human hepsin
MAQKEGGRTVPCCSRPKVAALTAGTLLLLTAIGAASWAIVAVLLRSDQEPLYPVQVSSADA
RLMVFDKTEGTWRLLCSSRSNARVAGLSCEEMGFLRALTHSELDVRTAGANGTSGFFCVD
EGRLPHTQRLLEVISVCDCPRGRFLAAICQDCGRRKLPVDRIVGGRDTSLGRWPWQVSLRY
DGAHLCGGSLLSGDWVLTAAHCFPERNRVLSRWRVFAGAVAQASPHGLQLGVQAVVYHG
GYLPFRDPNSEENSNDIALVHLSSPLPLTEYIQPVCLPAAGQALVDGKICTVTGWGNTQYYG
QQAGVLQEARVPIISNDVCNGADFYGNQIKPKMFCAGYPEGGIDACQGDSGGPFVCEDSISR
TPRWRLCGIVSWGTGCALAQKPGVYTKVSDFREWIFQAIKTHSEASGMVTQL
SEQ ID NO: 42: Original recombinant hepsin polynucleotide
sequence; c-terminal his tag was included for purification
purposes.
    1 cgggacacca gcttgggccg gtggccgtgg caagtcagcc ttcgctatga
  51 tggagcacac ctctgtgggg gatccctgct ctccggggac tgggtgctga
101 cagccgccca ctgcttcccg gagcggaacc gggtcctgtc ccgatggcga
151 gtgtttgccg gtgccgtggc ccaggcctct ccccacggtc tgcagctggg
201 ggtgcaggct gtggtctacc acgggggcta tcttcccttt cgggacccca
251 acagcgagga gaacagcaac gatattgccc tggtccacct ctccagtccc
301 ctgcccctca cagaatacat ccagcctgtg tgcctcccag ctgccggcca
351 ggccctggtg gatggcaaga tctgtaccgt gacgggctgg ggcaacacgc
401 agtactatgg ccaacaggcc ggggtactcc aggaggctcg agtccccata
451 atcagcaatg atgtctgcaa tggcgctgac ttctatggaa accagatcaa
501 gcccaagatg ttctgtgctg gctaccccga gggtggcatt gatgcctgcc
551 agggcgacag cggtggtccc tttgtgtgtg aggacagcat ctctcggacg
601 ccacgttggc ggctgtgtgg cattgtgagt tggggcactg gctgtgccct
651 ggcccagaag ccaggcgtct acaccaaagt cagtgacttc cgggagtgga
701 tcttccaggc cataaagact cactccgaag ccagcggcat ggtgacccag
751 ctc
SEQ ID NO: 43: Original recombinant hepsin amino acid
sequence; c-terminal his tag was included for purification
purposes.
    1 RDTSLGRWPW QVSLRYDGAH LCGGSLLSGD WVLTAAHCFP ERNRVLSRWR
  51 VFAGAVAQAS PHGLQLGVQA VVYHGGYLPF RDPNSEENSN DIALVHLSSP
101 LPLTEYIQPV CLPAAGQALV DGKICTVTGW GNTQYYGQQA GVLQEARVPI
151 ISNDVCNGAD FYGNQIKPKM FCAGYPEGGI DACQGDSGGP FVCEDSISRT
201 PRWRLCGIVS WGTGCALAQK PGVYTKVSDF REWIFQAIKT HSEASGMVTQ
251 L
SEQ ID NO: 44: Recombinant hepsin protein with thrombin
cleavage site underlined and c-terminal 6x-his-tag
(SEQ ID NO: 47) in italics
MLYPVQVSSADARLMVFDKTEGTWRLLCSSRSNARVAGLSCEEMGFLRALTHSELDVRTA
GANGTSGFFCVDEGRLPHTQRLLEVISVCDCPRGRFLAAICQDCGRRKLPVDRLVPRGSIVG
GRDTSLGRWPWQVSLRYDGAHLCGGSLLSGDWVLTAAHCFPERNRVLSRWRVFAGAVAQ
ASPHGLQLGVQAVVYHGGYLPFRDPNSEENSNDIALVHLSSPLPLTEYIQPVCLPAAGQALV
DGKICTVTGWGNTQYYGQQAGVLQEARVPIISNDVCNGADFYGNQIKPKMFCAGYPEGGI
DACQGDSGGPFVCEDSISRTPRWRLCGIVSWGTGCALAQKPGVYTKVSDFREWIFQAIKTH
SEASGMVTQLHHHHHH
SEQ ID NO: 45: Hepsin with FLAG tag underlined and a linker
in italics
MAQKEGGRTVPCCSRPKVAALTAGTLLLLTAIGAASWAIVAVLLRSDQEPLYPVQVSSADA
RLMVFDKTEGTWRLLCSSRSNARVAGLSCEEMGFLRALTHSELDVRTAGANGTSGFFCVD
EGRLPHTQRLLEVISVCDCPRGRFLAAICQDCGRRKLPVDRIVGGRDTSLGRWPWQVSLRY
DGAHLCGGSLLSGDWVLTAAHCFPERNRVLSRWRVFAGAVAQASPHGLQLGVQAVVYHG
GYLPFRDPNSEENSNDIALVHLSSPLPLTEYIQPVCLPAAGQALVDGKICTVTGWGNTQYYG
QQAGVLQEARVPIISNDVCNGADFYGNQIKPKMFCAGYPEGGIDACQGDSGGPFVCEDSISR
TPRWRLCGIVSWGTGCALAQKPGVYTKVSDFREWIFQAIKTHSEASGMVTQLGTGGGSGG
GSGGGASDYKDDDDK
Residues shown in orange represent the inserted linker and
residues shown in red represent the Flag tag.
SEQ ID NO: 46: Hepsin with thrombin cleavage site underlined,
a linker in italics, and a FLAG tag in underlined italics
MAQKEGGRTVPCCSRPKVAALTAGTLLLLTAIGAASWAIVAVLLRSDQEPLYPVQVSSADA
RLMVFDKTEGTWRLLCSSRSNARVAGLSCEEMGFLRALTHSELDVRTAGANGTSGFFCVD
EGRLPHTQRLLEVISVCDCPRGRFLAAICQDCGRRKLPVDRLVPRGSIVGGRDTSLGRWPW
QVSLRYDGAHLCGGSLLSGDWVLTAAHCFPERNRVLSRWRVFAGAVAQASPHGLQLGVQ
AVVYHGGYLPFRDPNSEENSNDIALVHLSSPLPLTEYIQPVCLPAAGQALVDGKICTVTGWG
NTQYYGQQAGVLQEARVPIISNDVCNGADFYGNQIKPKMFCAGYPEGGIDACQGDSGGPF
VCEDSISRTPRWRLCGIVSWGTGCALAQKPGVYTKVSDFREWIFQAIKTHSEASGMVTQLG
TGGGSGGGSGGGASDYKDDDDK
Residues shown in green represent the inserted thrombin
cleavage site, residues shown in orange represent the
inserted linker and residues in red represent the Flag tag.
SEQ ID NO: 47: 6x-histidine-tag
His- His- His- His- His- His
SEQ ID NO: 48: Extracellular domain (ECD) with Flag tag,
linker and gLUC signal sequence.
MGVKVLFALICIAVAEAGRDTSLGRWPWOVSLRYDGAHLCGGSLLSGDWVLTAAHCFPER
NRVLSRWRVFAGAVAQASPHGLQLGVQAVVYHGGYLPFRDPNSEENSNDIALVHLSSPLPL
TEYIQPVCLPAAGQALVDGKICTVTGWGNTQYYGQQAGVLQEARVPIISNDVCNGADFYG
NQIKPKMFCAGYPEGGIDACQGDSGGPFVCEDSISRTPRWRLCGIVSWGTGCALAQKPGVY
TKVSDFREWIFQAIKTHSEASGMVTQLGTGGGSGGGSGGGAS [symbol]
Underlined & italicized residues indicate Flag tag,
italicized residues indicate inserted linker and
underlined residues indicate gLUC signal sequence.
```

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application.

### Example 1: Establishment of Hybridomas

His-tagged recombinant hepsin protein having an amino acid sequence of SEQ ID NO: 43 was expressed and purified in an E. coli system. The above expressed protein was used to generate hybridomas.

Hybridomas of mouse origin that produce monoclonal antibodies, 2H2 and 5B8, were prepared using standard techniques following immunization of female BALB/c mice intraperitoneally with 25 μg of a recombinant hepsin (SEQ ID NO: 43) per mouse in Complete Freund's adjuvant (primary immunization; 125 μL and subsequent boosts) every 21 days, 25 μg immunogen (max injection volume 125 μL to a maximum of 5× injections) in Incomplete Freund's adjuvant. Splenocytes were isolated and hybridomas were prepared using techniques of PEG-1500 fusion technique.

The resulting antibodies were then screened against ascites fluid from ovarian cancer patients for recognition of native, circulating (shed) hepsin protein (data not shown).

The resulting antibodies were then further screened for their ability to produce a viable standard curve using the aforementioned E. coli produced recombinant protein as the standard protein by means of a double-determinant (1) immunoassay (“sandwich ELISA”).

An additional recombinant hepsin protein comprising the extracellular region (381 aa) was engineered via the addition of a thrombin cleavage site to mimic cell surface auto-activation of hepsin and to increase the storage half-life (SEQ ID NO: 44).

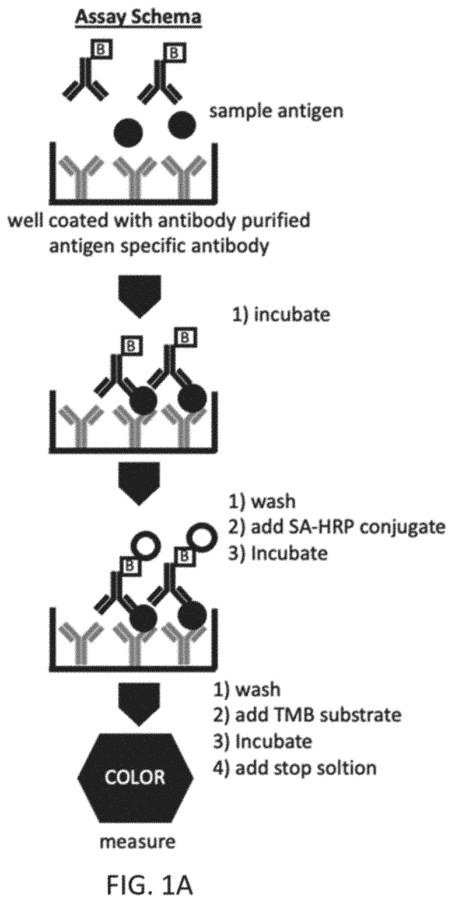

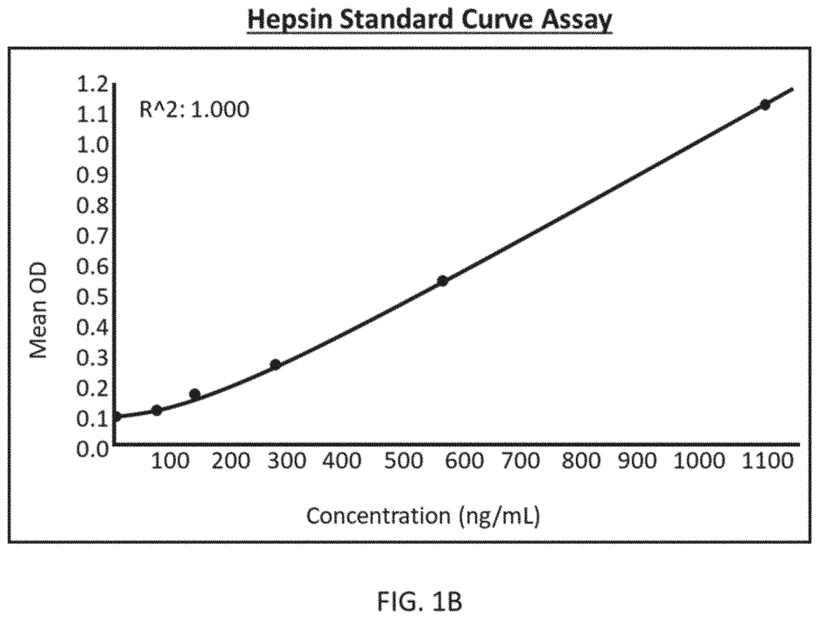

Following activation of SEQ ID NO: 44 with thrombin, the activated recombinant hepsin was used to re-screen mAb 2H2 and mAb 5B8 by sandwich ELISA, which also produced a viable standard curve (see, FIGS. 1A and 1B).

TABLE 7
Spike and Recovery Sample Data for Hepsin Assay

| Sample | Expected | Actual | Recovery |
|---|---|---|---|
| WB83017 | 500 | 509 | 102% |
| MH62217 | 500 | 464 | 93% |

This screening confirmed that the antibodies mAb 2H2 and mAb 5B8 were able to specifically bind to activated, soluble, c-terminal extracellular, circulating hepsin, but do not bind to the full-length zymogen.

### Example 2: Screening to Determine Specificity

Given that the c-terminus of the extracellular portion of hepsin exhibits homology to other members of the trypsin-like serine protease family, the antibodies were screened against a panel of recombinant serine proteases using an ELISA assay.

Multiple serine proteases and one matrix metalloproteinase assayed on the Hepsin ELISA assay to determine if any of them cross-reacted with the hepsin assay. The proteins assayed were recombinant proteins. The serine proteases Matripase, KLK6, KLK7, and KLK8 were assayed at 280 ng/ml, while the matrix metalloproteinase MMP-7 were assayed at 32 ng/ml. Each protein was assayed in eight (8) wells. Each protein recorded average levels below the 10 ng/ml sensitivity of the Hepsin assay. See, Table 8.

TABLE 8

| Spiked at 280 | Spiked at 280 | Spiked at 280 | Spiked at 280 | Spiked at 32 |
|---|---|---|---|---|
| ng/ml | ng/ml | ng/ml | ng/ml | ng/ml |
| Matriptase | KLK6 | KLK7 | KLK8 | MMP-7 |
| ng/ml | ng/ml | ng/ml | ng/ml | ng/ml |
| 4.56 | 4.56 | 3.28 | 5.19 | 3.93 |
| 3.28 | 3.28 | 1.20 | 4.56 | 1.20 |
| 2.62 | 1.93 | 1.20 | 4.56 | 1.20 |
| 3.28 | 1.93 | 1.93 | 3.93 | 0.41 |
| 2.62 | 23.79 | 1.20 | 5.80 | 0.41 |
| 3.28 | 1.93 | 2.62 | 3.93 | 1.93 |
| 4.56 | 1.93 | 3.28 | 8.18 | 2.62 |
| 3.93 | 4.56 | 5.80 | 2.62 | 1.93 |

The data demonstrated that antibodies are specific for the c-terminus of the extracellular portion of Hepsin and do not cross-react with other members of the trypsin-like serine protease family.

### Example 3: Assessment of Binding Affinity

To determine binding affinity in vitro, a sandwich ELISA was used to analyze circulating Hepsin levels occurring in human prostate cancer cells lysates, LNCaP, engineered to either overexpress or knockdown Hepsin (Table 9). Cell lysates from either Hepsin-overexpressing or Hepsin-depleted (knockdown), and vector controls were used. Lastly, antibody binding affinity to cell surface, activated Hepsin was performed via flow cytometry of mAb 2H2 and mAb 5B8, respectively, using HEK-293T cells overexpressing wild-type (wt)-Hepsin

TABLE 9

| Cell Line | Hepsin Level (ng/ml) |
|---|---|
| LNCaP Hepsin Overexpression | 3109.14 |
| LNCaP Hepsin Knockdown | 936.21 |
| LNCaP Vector Control | 2816.43 |

### Example 4: Detection of Extracellular Hepsin

This example describes an in vitro assay for detecting extracellular Hepsin and diagnosing a subject with having cancer utilizing an antibody or antigen-binding fragment described herein that specifically binds to the extracellular region of Hepsin.

In this example, the inventors reanalyzed pre-existing, publicly available gene expression datasets (2 datasets, 326 patient samples), which revealed a significant upregulation of Hepsin gene expression in tumor tissue versus benign tissue and further pronounced Hepsin upregulation in androgen-dependent metastatic lesions and androgen-independent metastatic lesions. To further examine the potential correlation of Hepsin overexpression to androgen-independence, we re-analyzed in vitro data comparing LNCaP parental versus androgen-independent LNCaP subclones. This analysis revealed a stepwise increase in Hepsin expression over the course of androgen deprivation, which was accompanied by a concomitant decrease in PSA expression. Lastly, to evaluate circulating Hepsin protein levels as they occur in patient serum, a total of 424 subjects suspected of prostate cancer were analyzed for circulating Hepsin levels from 2015 to 2018 at the time of biopsy and followed for recurrence or survival until the end of the study. Serum Hepsin levels, as determined by enzyme-linked immunosorbent assay, rendered a specificity of 89% (Gleason <6 (3+3); Hepsin positive, >100 ng/mL). Within this cohort, 38 patients presented with biochemical recurrence, of which 44% were Hepsin positive and exhibited a 22-month accelerated clinical path to recurrence versus Hepsin negative patients. Collectively, these results highlight the longitudinal diagnostic use of circulating Hepsin levels in the prostate cancer setting, including metastatic prostate cancer.

Exemplary Assay Protocols

A biological sample is obtained from a subject suspected of having prostate cancer, ovarian cancer, a carcinoma (e.g., renal cell carcinoma), a metastasis thereof (e.g., a metastatic prostate cancer, a metastatic ovarian cancer, a metastatic carcinoma such as a metastatic renal cell carcinoma), or a combination thereof.

The biological sample is optionally treated prior to use in the assay. The sample is then tested using, for example, an ELISA assay, ELISPOT, immunohistochemistry, or antibody adaptation to microbeads for multiplex and/or microfluidic platforms. In one example, results with mAb 2H2a, mAb 2H2b, or mAb 5B8, are compared to a control that contains a control antibody and/or a control that lacks sample.

Results are obtained and a cancer is detected when binding of the antibody is detected.

While certain embodiments of the present application have been shown and described herein, it will be obvious that such embodiments are provided by way of example only. Numerous variations, changes, and substitutions may occur to those skilled in the art without departing from the embodiments; it should be understood that various alternatives to the embodiments described herein may be employed in practicing the methods described herein.

1.-25. (canceled)

26. An antibody or antigen-binding fragment thereof that comprises:
a. a variable heavy chain, wherein the variable heavy chain comprises a reconstructed polypeptide consensus sequence selected from any one of SEQ ID NOS: 1-3; or
b. a variable light chain, wherein the variable light chain comprises a reconstructed polypeptide consensus sequence selected from any one of SEQ ID NOS: 4-5.

27. The antibody or antigen-binding fragment thereof of claim 26, wherein the antibody or antigen-binding fragment thereof selectively binds to circulating Hepsin or to the c-terminus of circulating Hepsin.

28. The antibody or antigen-binding fragment thereof of claim 26, wherein the antibody or antigen-binding fragment thereof does not selectively bind to serine proteases Matripase, KLK6, KLK7, and KLK8.

29. The antibody or antigen-binding fragment thereof of claim 27, wherein the Hepsin is human Hepsin.

30. The antibody or antigen-binding fragment thereof of claim 27, wherein the Hepsin is biologically-active extracellular Hepsin.

31. The antibody or antigen-binding fragment thereof of claim 27, wherein the circulating Hepsin comprises an amino acid sequence of SEQ ID NO: 44.

32. The antibody or antigen-binding fragment thereof of claim 27, wherein the circulating Hepsin comprises wild-type human Hepsin, and wherein the wild-type human Hepsin comprises an amino acid sequence of SEQ ID NO: 41.

33. The antibody or antigen-binding fragment thereof of claim 26, wherein the variable heavy chain comprises a complementarity-determining region CDR-H1, CDR-H2 and CDR-H3, wherein CDR-H1 comprises a reconstructed polypeptide consensus sequence selected from any one of SEQ ID NOS: 16 (KYWMS), 18 (SGYSWH), and 19 (SFGMH), CDR-H2 comprises a reconstructed polypeptide consensus sequence selected from any one of SEQ ID NOS: 11 (EINPDGSTIIYTPSLKD), 13 (YIHYNGNTNYNPSLKS), and 14 (YISSGSSAIYYADTVKG), and CDR-H3 comprises a reconstructed polypeptide consensus sequence selected from any one of SEQ ID NOS: 6 (RANWYYFDY), ADF, and 9 (SNWDYFDY).

34. The antibody or antigen-binding fragment thereof of claim 26, wherein the variable light chain comprises a complementarity-determining region CDR-L1, CDR-L2 and CDR-L3, wherein CDR-L1 comprises a reconstructed polypeptide consensus sequence selected from any one of SEQ ID NOS: 17 (TASSSVSSSNFH) and 20 (KSSQSLLNSRIRKNYLA), CDR-L2 comprises a reconstructed polypeptide consensus sequence selected from any one of SEQ ID NOS: 12 (STSNLAS) and 15 (WASTRES), and CDR-L3 comprises a reconstructed polypeptide consensus sequence selected from any one of SEQ ID NOS: 7 (HQYHRSPRT) and 10 (KQSYNLWT).

35. The antibody or antigen-binding fragment thereof of claim 26, wherein the variable heavy chain comprises a variable heavy chain CDR-H1, CDR-H2 and CDR-H3 selected from any one of SEQ ID NOS: 1-3, and wherein the CDR-H1, CDR-H2, and CDR-H3 are defined by Chothia numbering, Martin numbering, Kabat numbering, AHo numbering, or IMGT numbering.

36. The antibody or antigen-binding fragment thereof of claim 26, wherein the variable light chain comprises a variable light chain CDR-L1, CDR-L2 and CDR-L3 selected from any one of SEQ ID NOS: 4-5, and wherein the CDR-L1, CDR-L2, and CDR-L3 are defined by Chothia numbering, Martin numbering, Kabat numbering, AHo numbering, or IMGT numbering.

37. An isolated nucleic acid that comprises:
a. a reconstructed nucleic acid consensus sequence encoding a heavy chain polypeptide of an antibody, wherein the nucleic acid consensus sequence is selected from any one of SEQ ID NOS: 21-23; or
b. a reconstructed nucleic acid consensus sequence encoding a light chain polypeptide of an antibody, wherein the nucleic acid consensus sequence is selected from any one of SEQ ID NOS: 24-25.

38. A method of identifying the presence of circulating hepsin in a biological sample comprising:
a) contacting the biological sample with an antibody that selectively binds circulating hepsin; and
b) determining whether circulating hepsin is present in the biological sample.

39. The method of claim 38, wherein the antibody that selectively binds the circulating hepsin comprises:
a. a variable heavy chain, wherein the variable heavy chain comprises a reconstructed polypeptide consensus sequence selected from any one of SEQ ID NOS: 1-3; or
b. a variable light chain, wherein the variable light chain comprises a reconstructed polypeptide consensus sequence selected from any one of SEQ ID NOS: 4-5.

40. The method of claim 38, wherein the biological sample is a non-tissue sample.

41. The method of claim 38, wherein the method further comprises identifying presence of or risk of developing cancer in an individual.

42. The method of claim 38, wherein the method further comprises identifying risk of recurrence of cancer in an individual.

43. The method of claim 38, wherein the method further comprises identifying risk of metastasis of cancer in an individual.


SEQUENCE LISTING (49 sequences)

SEQ ID NO: 1 - protein, 136 aa, synthetic construct
  source 1..136: note = Description of Artificial Sequence: Synthetic polypeptide
MDFGLIFFIVALLKGVQCEVKLLESGGGLVQPGGSLKLSCAASGFDFNKYWMSWVRQAPGKGLEWIGEINPDGSTIIYTPSLKDKFIISRDNARNTLYLQMSKVRSEDTALYYCARRANWYYFDYWGQGTTLTVSS

SEQ ID NO: 2 - protein, 130 aa, synthetic construct
  source 1..130: note = Description of Artificial Sequence: Synthetic polypeptide
MRVLILLCLFTAFPGILSDVQLQESGPDLVKPSQSLSLTCTVTGYSITSGYSWHWIRQFPGNILEWMAYIHYNGNTNYNPSLKSRISITRDTSKNQFFLQLNSVTTEDTATYFCARADFWGQGTTLTVSS

SEQ ID NO: 3 - protein, 136 aa, synthetic construct
  source 1..136: note = Description of Artificial Sequence: Synthetic polypeptide
MDSRLNLVFLVLILKGVQCDVQLVESGGDLVQPGESRKLSCAASGFTFSSFGMHWVRQAPEKGLEWVAYISSGSSAIYYADTVKGRFTISRDNPKNTLFLHMTSLRSEDTAIYYCAGSNWDYFDYWGQGTTLTVSS

SEQ ID NO: 4 - protein, 130 aa, synthetic construct
  source 1..130: note = Description of Artificial Sequence: Synthetic polypeptide
MDFQVQIFSFLLISASVIMSRGQIVLTQSPAIMSASLGERVTMTCTASSSVSSSNFHWYQQKPGSSPKLWIYSTSNLASGVPTRFSGSGSGTSYSLTISSMEAEDAATYYCHQYHRSPRTFGGGTKLEIK

SEQ ID NO: 5 - protein, 132 aa, synthetic construct
  source 1..132: note = Description of Artificial Sequence: Synthetic polypeptide
MDSQAQVLILLLLWVSGTCEDIVMSQSPSSLAVSAGEKVTMSCKSSQSLLNSRIRKNYLAWYQQKPGQSPKLLIYWASTRESGVPDRFTGSGSGTDFTLTISSVQAEDLAVYYCKQSYNLWTFGGGTKLEIK

SEQ ID NO: 6 - protein, 9 aa, synthetic construct
  source 1..9: note = Description of Artificial Sequence: Synthetic peptide
RANWYYFDY

SEQ ID NO: 7 - protein, 9 aa, synthetic construct
  source 1..9: note = Description of Artificial Sequence: Synthetic peptide
HQYHRSPRT

SEQ ID NO: 8 - skipped

SEQ ID NO: 9 - protein, 8 aa, synthetic construct
  source 1..8: note = Description of Artificial Sequence: Synthetic peptide
SNWDYFDY

SEQ ID NO: 10 - protein, 8 aa, synthetic construct
  source 1..8: note = Description of Artificial Sequence: Synthetic peptide
KQSYNLWT

SEQ ID NO: 11 - protein, 17 aa, synthetic construct
  source 1..17: note = Description of Artificial Sequence: Synthetic peptide
EINPDGSTIIYTPSLKD

SEQ ID NO: 12 - protein, 7 aa, synthetic construct
  source 1..7: note = Description of Artificial Sequence: Synthetic peptide
STSNLAS

SEQ ID NO: 13 - protein, 16 aa, synthetic construct
  source 1..16: note = Description of Artificial Sequence: Synthetic peptide
YIHYNGNTNYNPSLKS

SEQ ID NO: 14 - protein, 17 aa, synthetic construct
  source 1..17: note = Description of Artificial Sequence: Synthetic peptide
YISSGSSAIYYADTVKG

SEQ ID NO: 15 - protein, 7 aa, synthetic construct
  source 1..7: note = Description of Artificial Sequence: Synthetic peptide
WASTRES

SEQ ID NO: 16 - protein, 5 aa, synthetic construct
  source 1..5: note = Description of Artificial Sequence: Synthetic peptide
KYWMS

SEQ ID NO: 17 - protein, 12 aa, synthetic construct
  source 1..12: note = Description of Artificial Sequence: Synthetic peptide
TASSSVSSSNFH

SEQ ID NO: 18 - protein, 6 aa, synthetic construct
  source 1..6: note = Description of Artificial Sequence: Synthetic peptide
SGYSWH

SEQ ID NO: 19 - protein, 5 aa, synthetic construct
  source 1..5: note = Description of Artificial Sequence: Synthetic peptide
SFGMH

SEQ ID NO: 20 - protein, 17 aa, synthetic construct
  source 1..17: note = Description of Artificial Sequence: Synthetic peptide
KSSQSLLNSRIRKNYLA

SEQ ID NO: 21 - DNA, 408 bp, synthetic construct
  source 1..408: mol_type = other DNA; note = Description of Artificial Sequence: Synthetic polynucleotide
atggattttgggctgattttttttattgttgctcttttaaaaggggtccagtgtgaggtgaaacttctcgagtctggaggtggcctggtgcagcctggaggatccctgaaactctcctgtgcagcctcaggattcgattttaataaatactggatgagttgggtccggcaggctccagggaaagggctagaatggattggagaaattaatccagatggcagtacgatcatctatacgccatctctaaaggataaattcatcatctccagagacaacgccagaaatactctgtacctgcaaatgagcaaagtgagatctgaggacacagccctttattactgtgcaagacgggctaactggtactactttgactactggggccaaggcaccactctcacagtctcctca

SEQ ID NO: 22 - DNA, 390 bp, synthetic construct
  source 1..390: mol_type = other DNA; note = Description of Artificial Sequence: Synthetic polynucleotide
atgagagtgctgattcttttgtgcctgttcacagcctttcctggtatcctgtctgatgtgcagcttcaggagtcaggacctgacctggtgaaaccttctcagtcactttcactcacctgcactgtcactggctactccatcaccagtggttatagctggcactggatccggcagtttccaggaaacatactggaatggatggcctacatacactacaatggtaacactaactacaacccatctctcaaaagtcggatctctatcactcgagacacctccaagaaccagttcttcctgcagttgaattctgtgactactgaggacactgccacatatttctgtgcaagagccgatttctggggccaaggcaccactctcacagtctcctca

SEQ ID NO: 23 - DNA, 408 bp, synthetic construct
  source 1..408: mol_type = other DNA; note = Description of Artificial Sequence: Synthetic polynucleotide
atggactccaggctcaatttagttttccttgtccttattttaaaaggtgtccagtgtgatgtgcagctggtggagtctgggggagacttagtgcagcctggagagtcccggaaactctcctgtgcagcctctggattcactttcagtagctttggaatgcactgggttcgtcaggctccagagaaggggctggagtgggtcgcatacattagtagtggcagtagtgccatctactatgcagacacagtgaagggccgattcaccatctccagagacaatcccaagaacaccctgttcctacacatgaccagtctaaggtctgaggacacggccatatattactgtgcaggaagcaactgggactactttgactactggggccaaggcaccactctcacagtctcctca

SEQ ID NO: 24 - DNA, 390 bp, synthetic construct
  source 1..390: mol_type = other DNA; note = Description of Artificial Sequence: Synthetic polynucleotide
atggattttcaggtgcagattttcagcttcctgctaatcagtgcctcagtcataatgtccagaggacaaattgttctcacccagtctccagcaatcatgtctgcatctctaggggaacgggtcaccatgacctgcactgccagctcaagtgtaagttccagtaactttcactggtaccagcagaagccaggatcctcccccaaactctggatttatagcacctccaacctggcttctggagtcccaactcgcttcagtggcagtgggtctgggacctcttactctctcacaatcagcagcatggaggctgaagatgctgccacttattactgccaccagtatcatcgttccccacggacgttcggtggaggcaccaagctggaaatcaaa

SEQ ID NO: 25 - DNA, 396 bp, synthetic construct
  source 1..396: mol_type = other DNA; note = Description of Artificial Sequence: Synthetic polynucleotide
atggattcacaggcccaggttcttatattgctgctgctatgggtatctggtacctgtgaggacattgtgatgtcacagtctccatcctccctggctgtgtcagcaggagagaaggtcactatgagctgcaaatccagtcagagtctgctaaacagtagaatccgaaagaactacttggcttggtaccagcagaaaccagggcagtctcctaaattgttgatctactgggcatccactagggaatctggggtccctgatcgcttcacaggcagtggatctgggacagatttcactctcaccatcagcagtgtgcaggctgaggacctggcagtttattactgcaaacaatcttataatctgtggacgttcggtggaggcaccaagctggaaatcaaa

SEQ ID NO: 26 - DNA, 27 bp, synthetic construct
  source 1..27: mol_type = other DNA; note = Description of Artificial Sequence: Synthetic oligonucleotide
cgggctaactggtactactttgactac

SEQ ID NO: 27 - skipped

SEQ ID NO: 28 - DNA, 24 bp, synthetic construct
  source 1..24: mol_type = other DNA; note = Description of Artificial Sequence: Synthetic oligonucleotide
agcaactgggactactttgactac

SEQ ID NO: 29 - DNA, 27 bp, synthetic construct
  source 1..27: mol_type = other DNA; note = Description of Artificial Sequence: Synthetic oligonucleotide
caccagtatcatcgttccccacggacg

SEQ ID NO: 30 - DNA, 24 bp, synthetic construct
  source 1..24: mol_type = other DNA; note = Description of Artificial Sequence: Synthetic oligonucleotide
aaacaatcttataatctgtggacg

SEQ ID NO: 31 - DNA, 51 bp, synthetic construct
  source 1..51: mol_type = other DNA; note = Description of Artificial Sequence: Synthetic oligonucleotide
gaaattaatccagatggcagtacgatcatctatacgccatctctaaaggat

SEQ ID NO: 32 - DNA, 48 bp, synthetic construct
  source 1..48: mol_type = other DNA; note = Description of Artificial Sequence: Synthetic oligonucleotide
tacatacactacaatggtaacactaactacaacccatctctcaaaagt

SEQ ID NO: 33 - DNA, 51 bp, synthetic construct
  source 1..51: mol_type = other DNA; note = Description of Artificial Sequence: Synthetic oligonucleotide
tacattagtagtggcagtagtgccatctactatgcagacacagtgaagggc

SEQ ID NO: 34 - DNA, 21 bp, synthetic construct
  source 1..21: mol_type = other DNA; note = Description of Artificial Sequence: Synthetic oligonucleotide
agcacctccaacctggcttct

SEQ ID NO: 35 - DNA, 24 bp, synthetic construct
  source 1..24: mol_type = other DNA; note = Description of Artificial Sequence: Synthetic oligonucleotide
aaacaatcttataatctgtggacg

SEQ ID NO: 36 - DNA, 15 bp, synthetic construct
  source 1..15: mol_type = other DNA; note = Description of Artificial Sequence: Synthetic oligonucleotide
aaatactggatgagt

SEQ ID NO: 37 - DNA, 18 bp, synthetic construct
  source 1..18: mol_type = other DNA; note = Description of Artificial Sequence: Synthetic oligonucleotide
agtggttatagctggcac

SEQ ID NO: 38 - DNA, 15 bp, synthetic construct
  source 1..15: mol_type = other DNA; note = Description of Artificial Sequence: Synthetic oligonucleotide
agctttggaatgcac

SEQ ID NO: 39 - DNA, 36 bp, synthetic construct
  source 1..36: mol_type = other DNA; note = Description of Artificial Sequence: Synthetic oligonucleotide
actgccagctcaagtgtaagttccagtaactttcac

SEQ ID NO: 40 - DNA, 51 bp, synthetic construct
  source 1..51: mol_type = other DNA; note = Description of Artificial Sequence: Synthetic oligonucleotide
aaatccagtcagagtctgctaaacagtagaatccgaaagaactacttggct

SEQ ID NO: 41 - protein, 417 aa, Homo sapiens
MAQKEGGRTVPCCSRPKVAALTAGTLLLLTAIGAASWAIVAVLLRSDQEPLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDRIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQL

SEQ ID NO: 42 - DNA, 753 bp, synthetic construct
  source 1..753: mol_type = other DNA; note = Description of Artificial Sequence: Synthetic polynucleotide
cgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctc

SEQ ID NO: 43 - protein, 251 aa, synthetic construct
  source 1..251: note = Description of Artificial Sequence: Synthetic polypeptide
RDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQL

SEQ ID NO: 44 - protein, 380 aa, synthetic construct
  source 1..380: note = Description of Artificial Sequence: Synthetic polypeptide
MLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDRLVPRGSIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQLHHHHHH

SEQ ID NO: 45 - protein, 440 aa, synthetic construct
  source 1..440: note = Description of Artificial Sequence: Synthetic polypeptide
MAQKEGGRTVPCCSRPKVAALTAGTLLLLTAIGAASWAIVAVLLRSDQEPLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDRIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQLGTGGGSGGGSGGGASDYKDDDDK

SEQ ID NO: 46 - protein, 446 aa, synthetic construct
  source 1..446: note = Description of Artificial Sequence: Synthetic polypeptide
MAQKEGGRTVPCCSRPKVAALTAGTLLLLTAIGAASWAIVAVLLRSDQEPLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDRLVPRGSIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQLGTGGGSGGGSGGGASDYKDDDDK

SEQ ID NO: 47 - protein, 6 aa, synthetic construct
  source 1..6: note = Description of Artificial Sequence: Synthetic 6xHis tag
HHHHHH

SEQ ID NO: 48 - protein, 292 aa, synthetic construct
  source 1..292: note = Description of Artificial Sequence: Synthetic polypeptide
MGVKVLFALICIAVAEAGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQLGTGGGSGGGSGGGASDYKDDDDK

SEQ ID NO: 49 - protein, 15 aa, synthetic construct
  source 1..15: note = Description of Artificial Sequence: Synthetic peptide
GGGGSGGGGSGGGGS